(19) United States
(12) Patent Application Publication
(10) Pub. No.: US 2026/0294876 A1
(43) Pub. Date: Oct. 1, 2026

(54) DOSAGE REGIME OF CRINECERFONT FOR TREATING CONGENITAL ADRENAL HYPERPLASIA IN A PEDIATRIC SUBJECT

(71) Applicant: Neurocrine Biosciences, Inc., San Diego, CA (US)

(72) Inventors: Jean L. Chan, San Diego, CA (US); Robert H. Farber, San Diego, CA (US)

(21) Appl. No.: 19/482,565

(22) PCT Filed: May 7, 2024
(86) PCT No.: PCT/US2024/028110

§ 371 Date: Nov. 7, 2025

Related U.S. Application Data
(60) Provisional application No. 63/464,697, filed on May 8, 2023.

(51) Int. Cl. A61K 31/426; A61K 31/573; A61P 5/38

(52) U.S. Cl. CPC A61K 31/426; A61K 31/573; A61P 5/38

(57) ABSTRACT

Provided are methods of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof, in a pediatric subject having congenital adrenal hyperplasia.

### CROSS-REFERENCE TO RELATED APPLICATIONS

The present application claims the benefit of U.S. Provisional Application No. 63/464,697, filed May 8, 2023, which is incorporated herein by reference in its entirety.

### TECHNICAL FIELD

The present application is directed to methods of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof, in a pediatric subject having congenital adrenal hyperplasia.

### BACKGROUND OF THE INVENTION

Congenital adrenal hyperplasia (CAH) is a disease that includes a group of autosomal recessive disorders that result in an enzyme deficiency that alters the production of adrenal steroids due to 21-hydroxylase deficiency, a condition that results in little or no cortisol biosynthesis. One clinical manifestation of the absence of cortisol is the lack of feedback inhibition of pituitary adrenocorticotropic hormone (ACTH) secretion. Increased ACTH levels cause adrenal hyperplasia and the enzyme mutation causes a shunting of cortisol precursor steroids to alternate pathways. Most notably, the shunting of androgens leads to virilization and other developmental complications in females and the over-accumulation of ACTH is associated with the formation of testicular adrenal rest tumors in males. In addition, since the same enzyme (21-hydroxylase) is used in the pathway for the biosynthesis of the mineralocorticoids, a number of these patients suffer from aldosterone deficiency which can result in dehydration and death due to salt-wasting. The prevalence of classic 21-hydroxylase deficiency CAH in the US general population, based on newborn screening, has been documented as 1:10,000 to 1:20,800.

Pediatric patients from birth through adolescence, and females in particular, appear to be the most vulnerable population of CAH sufferers and represent the subgroup of patients with the greatest unmet medical need. Excessive androgen production in these younger patients results in early onset puberty and adrenarche, changes in skeletal maturation patterns, short stature caused by premature growth plate fusion, as well as significant hirsutism and acne problems. While survival is properly ensured through steroid replacement strategies based on physiologic dosing of glucocorticoids (e.g., hydrocortisone) and mineralocorticoids (e.g., fludrocortisone), these doses are often inadequate to suppress the accumulating ACTH and overproduction of progestogens and androgens (e.g., 17-hydroxyprogesterone [17-OHP], androstenedione, and testosterone). The uncontrolled symptoms of androgen excess, indeed, have a substantial impact on the day-to-day functioning and development of these patients.

Currently, exogenous corticosteroids are the standard of care for treating patients with classic CAH. This treatment is used to correct the cortisol deficiency and reduce the excessive ACTH levels and androgen excess. However, the dose and duration of steroid use required to suppress ACTH are typically well above the normal physiological level used for cortisol replacement alone (as in patients with Addison's disease). This increased exposure to glucocorticoids can lead to iatrogenic Cushing's syndrome, increased cardiovascular risk factors, glucose intolerance, reduced growth velocity, and decreased bone mineral density in CAH patients.

It has been demonstrated in clinical trials that orally active compounds that block CRF1, such as 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine (Compound A), provide a reduction from baseline in 17-hydroxyprogesterone (17-OHP) and androstenedione levels in amounts believed to allow use of lower, more physiologic doses of glucocorticoid (e.g., hydrocortisone) in patients with CAH. The structure of Compound A is shown below:

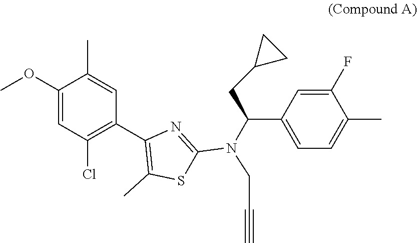

There is a significant, unmet need for methods for treating CAH. The present disclosure fulfills these and other needs, as evident in reference to the following disclosure.

### SUMMARY

Provided is a method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

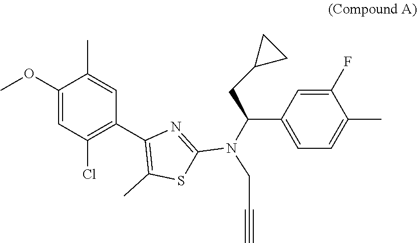

as an adjunct to glucocorticoid therapy in a pediatric subject having congenital adrenal hyperplasia (CAH), the method comprising: administering daily to the subject Compound A, or a pharmaceutically acceptable salt thereof, in two doses consisting of a first dose and a second dose wherein the second dose is greater than the first dose.

Further provided is a method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

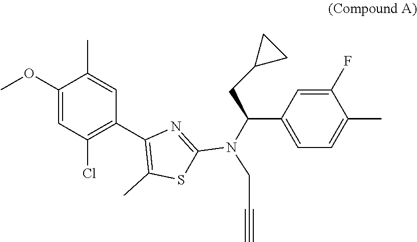

as an adjunct to glucocorticoid therapy in a pediatric subject having congenital adrenal hyperplasia (CAH), the method comprising: administering to the subject a first amount of Compound A, or a pharmaceutically acceptable salt thereof, for a first period of time, and subsequently administering to the subject a second amount of Compound A, or a pharmaceutically acceptable salt thereof, for a second period of time, wherein the second amount is administered in two doses consisting of a first dose and a second dose wherein the second dose is greater than the first dose.

Also provided is a method of treating congenital adrenal hyperplasia (CAH) in a pediatric subject already being treated with glucocorticoid therapy, the method comprising: administering daily to the subject 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

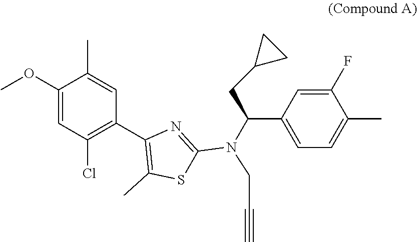

in two doses consisting of a first dose and a second dose wherein the second dose is greater than the first dose.

Also provided is a method of treating congenital adrenal hyperplasia (CAH) in a pediatric subject already being treated with glucocorticoid therapy, the method comprising: administering to the subject a first amount of 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

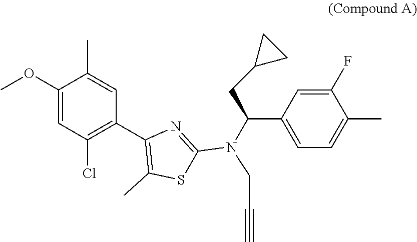

for a first period of time, and subsequently administering to the subject a second amount of Compound A, or a pharmaceutically acceptable salt thereof, for a second period of time, wherein the second amount is administered in two doses consisting of a first dose and a second dose wherein the second dose is greater than the first dose.

Further provided is a compound which is 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine:

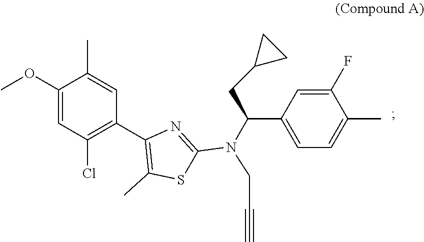

or a pharmaceutically acceptable salt thereof;
for use in a method of treating congenital adrenal hyperplasia (CAH) in a pediatric subject already being treated with glucocorticoid therapy, the method comprising:
   (1) administering to the subject a first amount of Compound A, or a pharmaceutically acceptable salt thereof, for a first period of time, and
   (2) subsequently administering to the subject a second amount of Compound A, or a pharmaceutically acceptable salt thereof, for a second period of time, wherein the second amount is administered in two doses consisting of a first dose and a second dose wherein the second dose is greater than the first dose.

Further provided is the use of a compound which is 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine:

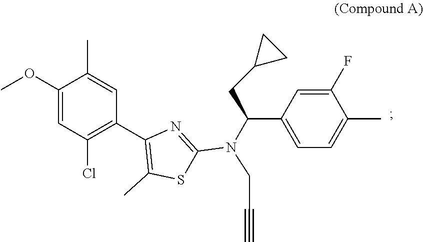

or a pharmaceutically acceptable salt thereof;
in the manufacture of a medicament for treating congenital adrenal hyperplasia (CAH) in a pediatric subject already being treated with glucocorticoid therapy, by a method comprising the steps:
   (1) administering to the subject a first amount of Compound A, or a pharmaceutically acceptable salt thereof, for a first period of time, and
   (2) subsequently administering to the subject a second amount of Compound A, or a pharmaceutically acceptable salt thereof, for a second period of time, wherein the second amount is administered in two doses consisting of a first dose and a second dose wherein the second dose is greater than the first dose.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, the headings provided herein are for convenience only and do not interpret the scope or meaning of the claimed invention.

Reference throughout this specification to “one embodiment” or “an embodiment” or “some embodiments” or “a certain embodiment” means that a particular feature, structure, or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, in some embodiments, “pharmaceutically acceptable salt” refers to acid addition salts with an inorganic or an organic acid. Lists of suitable salts are found in WO 87/05297, Johnston et al., published Sep. 11, 1987; Remington's Pharmaceutical Sciences, 17th ed., Mack Publishing Company, Easton, Pa., 1985, p. 1418; and J. Pharm. Sci., 66, 2 (1977), each of which is incorporated herein by reference in its entirety. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002, which is incorporated herein by reference in its entirety. The organic or inorganic acids include, but are not limited to, hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and the like. In some embodiments, “pharmaceutically acceptable salt” refers to base addition salts with an inorganic or an organic base. Inorganic bases which may be used to prepare salts include, but are not limited to, for example, sodium, potassium, lithium, ammonium, calcium, magnesium, iron, zinc, manganese, aluminum hydroxides, carbonates, bicarbonates, phosphates, and the like. Organic bases from which may be used to prepare salts include, but are not limited to, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

As used herein, “about” means±20% of the stated value, and includes more specifically values of ±10%, ±5%, ±2%, and ±1% of the stated value.

As used herein, “baseline” refers to the period of time just prior to initiation of therapy. The patient's condition just prior to initiation of therapy can be referred to as the patient's baseline condition.

As used herein, “adjusting administration”, “altering administration”, “adjusting dosing”, or “altering dosing” are all equivalent and mean tapering off, reducing, or increasing the dose of the substance, ceasing to administer the substance to the patient, or substituting a different active agent for the substance.

As used herein, “administering to a patient” refers to the process of introducing a composition or dosage form into the patient via an art-recognized means of introduction.

As used herein, “b.i.d.” refers to twice daily. In some embodiments, the twice daily doses are separated by about 12 hours.

As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

As used here, “congenital adrenal hyperplasia” or “CAH” refers to a group of genetic disorders that affect the adrenal glands. Congenital adrenal hyperplasia (CAH) is a group of autosomal recessive genetic disorders that result in little or no cortisol biosynthesis. One clinical manifestation of the absence of cortisol is the lack of feedback inhibition on CRF which causes dysregulation of the HPA axis. The most frequent form of the disease is 21-hydroxylase deficiency caused by mutations in the CYP21A2 gene located on chromosome 6p21, which accounts for approximately 95% of CAH cases, the 21-hydroxylase enzyme deficiency also causes a shunting of cortisol precursor steroids leading to excess androgen (e.g., 17-hydroxyprogesterone, androstenedione, and testosterone) product. These mutations can range from complete loss of enzyme activity required for synthesis of cortisol in the adrenal cortex to a spectrum of partial loss, which results in disease severity that is a direct consequence of a specific mutation. This continuum of 21-hydroxylase deficiency has been broadly classified into salt-wasting and simple-virilizing forms, grouped as classic (or classical) CAH, and the milder form known as non-classic (or non-classical) CAH (NCCAH) or “late-onset” CAH, which is usually diagnosed in late childhood or early adulthood. Non-classic CAH patients are either homozygous or compound heterozygotes, often with a classical CAH allele. These patients have sufficient enzyme activity (>20-50% of normal) such that they do not have salt-wasting or cortisol deficiency and have normal genitalia at birth, and many remain asymptomatic throughout life. In the less frequent form of the disease, which accounts for 5% of cases, mutation of the 11β-hydroxylase gene CYP11B1 results in CAH (11β-OH CAH). Classic CAH is a rare disease; the incidence of classic CAH is estimated to be ~1:15,000 worldwide. Approximately 75% of patients with classic CAH have the salt wasting form of the disease, characterized by severe enzyme deficiency and insufficient levels of cortisol and aldosterone. Approximately 25% of patients with classic CAH have the simple virilizing form, in which cortisol synthesis is impaired, but enzyme activity is sufficient for adequate production of aldosterone.

As used herein the term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

As used herein, a “dose” means the measured quantity of an active agent to be taken at one time by a patient. In certain embodiments, wherein the active agent is not Compound A free base, the quantity is the molar equivalent to the corresponding amount of Compound A free base. For example, often a drug is packaged in a pharmaceutically acceptable salt form, and the dosage for strength refers to the mass of the molar equivalent of the corresponding free base, Compound A.

As used herein, “dosing regimen” means the dose of an active agent taken at a first time by a patient and the interval (time or symptomatic) at which any subsequent doses of the active agent are taken by the patient such as from about 100 mg to about 300 mg once daily or from about 20 mg to about 160 mg twice daily, e.g., about 100 mg, about 150 mg, about 200 mg, about 250 mg, or about 300 mg once daily; or about 20 mg, about 40 mg, about 60 mg, about 80 mg, about 100 mg, about 120 mg, or about 160 mg twice daily. The additional doses of the active agent can be different from the dose taken at the first time. For example, a first dose may range from about 20 mg to about 160 mg, e.g., about 20 mg, about 40 mg, about 60 mg, about 80 mg, about 100 mg, about 120 mg, or about 160 mg; and a second dose may range from about 100 mg to about 300 mg, e.g., about 100 mg, about 150 mg, about 200 mg, about 250 mg, or about 300 mg.

As used herein, a “dosage” is the prescribed administration of a specific amount, number, and frequency of doses over a specific period of time.

As used herein, “effective amount” and “therapeutically effective amount” of an agent, compound, drug, composition, or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, “a medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, “Medication Guide” means an FDA-approved patient labeling for a pharmaceutical product conforming to the specifications as set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, “patient” or “individual” or “subject” means a mammal, including a human, for whom or which therapy is desired, and generally refers to the recipient of the therapy.

As used herein, “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pediatric” refers to a subject who is about 17 years of age or younger (i.e., to birth). In some embodiments, the pediatric subject is an adolescent, i.e., a subject who is from about 12 to about 17 years of age. In some embodiments, the pediatric subject is a child, i.e., a subject who is from about 2 years to less than about 12 years of age. In some embodiments, the pediatric subject is an infant, i.e., a subject who is from about 1 month to about 2 years of age. In some embodiments, the pediatric subject is a neonate, i.e., a subject who is about 1 month of age or younger (i.e., to birth).

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree.

As used herein, the term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is well known in the art. Except insofar as any conventional media or agent is incompatible with the active ingredient, its use in the therapeutic formulations is contemplated. Supplementary active ingredients can also be incorporated into the formulations. In addition, various excipients, such as are commonly used in the art, can be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example, indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “q.A.M.” means once daily in the morning.

As used herein, “q.P.M.” means once daily in the evening.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

Provided is a method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

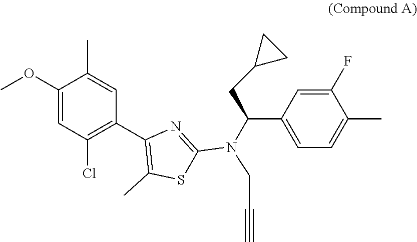

as an adjunct to glucocorticoid therapy in a pediatric subject having congenital adrenal hyperplasia (CAH), the method comprising: administering daily to the subject Compound A, or a pharmaceutically acceptable salt thereof, in two doses consisting of a first dose and a second dose wherein the second dose is greater than the first dose.

Also provided is a method of treating congenital adrenal hyperplasia (CAH) in a pediatric subject already being treated with glucocorticoid therapy, the method comprising: administering daily to the subject 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

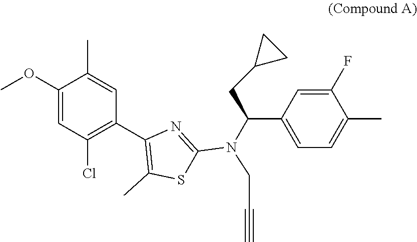

in two doses consisting of a first dose and a second dose wherein the second dose is greater than the first dose.

In some embodiments, administering Compound A, or a pharmaceutically acceptable salt thereof, refers to administration of Compound A, or a pharmaceutically acceptable salt thereof, with food. In some embodiments, Compound A, or a pharmaceutically acceptable salt thereof, is administered with food. In some embodiments, the administration of Compound A, or a pharmaceutically acceptable salt thereof, with food exhibits a positive food effect. In some embodiments, an increased, or positive, food effect is observed when Compound A, or a pharmaceutically acceptable salt thereof is administered to a subject in a fed state. In some embodiments, administration of Compound A, or a pharmaceutically acceptable salt thereof, results in an increased, or positive, food effect, whereby an increased Cₘₐₓ and/or AUC are observed when administered orally in the fed state as compared to the fasting state.

In some embodiments, administering Compound A, or a pharmaceutically acceptable salt thereof, refers to administration of Compound A, or a pharmaceutically acceptable salt thereof, with the subject's breakfast and evening meal.

In some embodiments, the subject weighs greater than or equal to about 55 kg.

In some embodiments, the first dose is equivalent to about 100 mg Compound A free base q.A.M. In some embodiments, the first dose is equivalent to about 0 mg Compound A free base q.A.M.

In some embodiments, the second dose is equivalent to about 200 mg Compound A free base q.P.M. In some embodiments, the second dose is equivalent to about 300 mg Compound A free base q.P.M.

In some embodiments, the subject weighs greater than or equal to about 55 kg and the first dose is equivalent to about 100 mg Compound A free base q.A.M. In some embodiments, the subject weighs greater than or equal to about 55 kg and the first dose is equivalent to about 0 mg Compound A free base q.A.M. In some embodiments, the subject weighs greater than or equal to about 55 kg and the second dose is equivalent to about 200 mg Compound A free base q.P.M. In some embodiments, the subject weighs greater than or equal to about 55 kg and the second dose is equivalent to about 300 mg Compound A free base q.P.M. In some embodiments, the subject weighs greater than or equal to about 55 kg, the first dose is equivalent to about 100 mg Compound A free base q.A.M. and the second dose is equivalent to about 200 mg Compound A free base q.P.M. In some embodiments, the subject weighs greater than or equal to about 55 kg, the first dose is equivalent to about 0 mg Compound A free base q.A.M. and the second dose is equivalent to about 200 mg Compound A free base q.P.M. In some embodiments, the subject weighs greater than or equal to about 55 kg, the first dose is equivalent to about 0 mg Compound A free base q.A.M. and the second dose is equivalent to about 300 mg Compound A free base q.P.M.

In some embodiments, prior to the administering of a first dose equivalent to about 0 mg Compound A free base q.A.M. and a second dose equivalent to about 200 mg Compound A free base q.P.M., the subject that weighs greater than or equal to about 55 kg demonstrated adequate disease control and was being administered a first dose equivalent to about 100 mg Compound A free base q.A.M. and a second dose equivalent to about 200 mg Compound A free base q.P.M. In some embodiments, prior to the administering of a first dose equivalent to about 0 mg Compound A free base q.A.M. and a second dose equivalent to about 200 mg Compound A free base q.P.M., the subject that weighs greater than or equal to about 55 kg demonstrated adequate disease control and was being administered a first dose equivalent to about 100 mg Compound A free base q.A.M. and a second dose equivalent to about 200 mg Compound A free base q.P.M. and a glucocorticoid at a target dose.

In some embodiments, prior to the administering of a first dose equivalent to about 0 mg Compound A free base q.A.M. and a second dose equivalent to about 300 mg Compound A free base q.P.M., the subject that weighs greater than or equal to about 55 kg demonstrated adequate disease control and was being administered a first dose equivalent to about 100 mg Compound A free base q.A.M. and a second dose equivalent to about 200 mg Compound A free base q.P.M. In some embodiments, prior to the administering of a first dose equivalent to about 0 mg Compound A free base q.A.M. and a second dose equivalent to about 300 mg Compound A free base q.P.M., the subject that weighs greater than or equal to about 55 kg demonstrated adequate disease control and was being administered a first dose equivalent to about 100 mg Compound A free base q.A.M. and a second dose equivalent to about 200 mg Compound A free base q.P.M. and a glucocorticoid at a target dose.

In some embodiments, the subject demonstrated adequate disease control if their androstenedione levels are ≤120% of the subject's baseline or ≤upper limit of normal (ULN) for age and sex of the subject.

In some embodiments, the subject demonstrated adequate disease control in the opinion of the investigator.

In some embodiments, the subject weighs greater than or equal to about 55 kg and prior to administration of Compound A, or a pharmaceutically acceptable salt thereof, in two doses consisting of a first dose and a second dose wherein the second dose is greater than the first dose, the subject had been administered Compound A, or a pharmaceutically acceptable salt thereof, as a twice daily dose, each dose having an equivalent amount of Compound A free base for a first period of time. In some embodiments, each dose is in an amount equivalent to about 100 mg of Compound A free base.

In some embodiments, the subject weighs from about 20 kg to about 55 kg.

In some embodiments, the first dose is in an amount equivalent to about 50 mg Compound A free base. In some embodiments, the first dose is in an amount equivalent to about 0 mg Compound A free base.

In some embodiments, the second dose is in an amount equivalent to about 100 mg Compound A free base. In some embodiments, the second dose is in an amount equivalent to about 150 mg Compound A free base.

In some embodiments, the subject weighs from about 20 kg to about 55 kg and the first dose is in an amount equivalent to about 50 mg Compound A free base. In some embodiments, the subject weighs from about 20 kg to about 55 kg and the first dose is in an amount equivalent to about 0 mg Compound A free base. In some embodiments, the subject weighs from about 20 kg to about 55 kg and the second dose is in an amount equivalent to about 100 mg Compound A free base. In some embodiments, the subject weighs from about 20 kg to about 55 kg and the second dose is in an amount equivalent to about 150 mg Compound A free base. In some embodiments, the subject weighs from about 20 kg to about 55 kg, the first dose is in an amount equivalent to about 50 mg Compound A free base, and the second dose is in an amount equivalent to about 100 mg Compound A free base. In some embodiments, the subject weighs from about 20 kg to about 55 kg, the first dose is in an amount equivalent to about 0 mg Compound A free base, and the second dose is in an amount equivalent to about 100 mg Compound A free base. In some embodiments, the subject weighs from about 20 kg to about 55 kg, the first dose is in an amount equivalent to about 0 mg Compound A free base, and the second dose is in an amount equivalent to about 150 mg Compound A free base.

In some embodiments, prior to the administering of a first dose equivalent to about 0 mg Compound A free base q.A.M. and a second dose equivalent to about 100 mg Compound A free base q.P.M., the subject that weighs from about 20 kg to about 55 kg demonstrated adequate disease control and was being administered a first dose equivalent to about 50 mg Compound A free base q.A.M. and a second dose equivalent to about 100 mg Compound A free base q.P.M. In some embodiments, prior to the administering of a first dose equivalent to about 0 mg Compound A free base q.A.M. and a second dose equivalent to about 100 mg Compound A free base q.P.M., the subject that weighs from about 20 kg to about 55 kg demonstrated adequate disease control and was being administered a first dose equivalent to about 50 mg Compound A free base q.A.M. and a second dose equivalent to about 100 mg Compound A free base q.P.M. and a glucocorticoid at a target dose.

In some embodiments, prior to the administering of a first dose equivalent to about 0 mg Compound A free base q.A.M. and a second dose equivalent to about 150 mg Compound A free base q.P.M., the subject that weighs from about 20 kg to about 55 kg demonstrated adequate disease control and was being administered a first dose equivalent to about 50 mg Compound A free base q.A.M. and a second dose equivalent to about 100 mg Compound A free base q.P.M. In some embodiments, prior to the administering of a first dose equivalent to about 0 mg Compound A free base q.A.M. and a second dose equivalent to about 150 mg Compound A free base q.P.M., the subject that weighs from about 20 kg to about 55 kg demonstrated adequate disease control and was being administered a first dose equivalent to about 50 mg Compound A free base q.A.M. and a second dose equivalent to about 100 mg Compound A free base q.P.M. and a glucocorticoid at a target dose.

In some embodiments, the subject demonstrated adequate disease control if their androstenedione levels are ≤120% of the subject's baseline or ≤upper limit of normal (ULN) for age and sex of the subject.

In some embodiments, the subject demonstrated adequate disease control in the opinion of the investigator.

In some embodiments, the subject weighs from about 20 kg to about 55 kg and prior to administration of Compound A, or a pharmaceutically acceptable salt thereof, in two doses consisting of a first dose and a second dose wherein the second dose is greater than the first dose, the subject had been administered Compound A, or a pharmaceutically acceptable salt thereof, as a twice daily dose, each dose having an equivalent amount of Compound A free base for a first period of time. In some embodiments, each dose is in an amount equivalent to about 50 mg of Compound A free base.

In some embodiments, the subject weighs from about 10 kg to about 20 kg.

In some embodiments, the first dose is in an amount equivalent to about 25 mg Compound A free base. In some embodiments, the first dose is in an amount equivalent to about 0 mg Compound A free base.

In some embodiments, the second dose is in an amount equivalent to about 50 mg Compound A free base. In some embodiments, the second dose is in an amount equivalent to about 75 mg Compound A free base.

In some embodiments, the subject weighs from about 10 kg to about 20 kg and the first dose is in an amount equivalent to about 25 mg Compound A free base. In some embodiments, the subject weighs from about 10 kg to about 20 kg and the first dose is in an amount equivalent to about 0 mg Compound A free base. In some embodiments, the subject weighs from about 10 kg to about 20 kg and the second dose is in an amount equivalent to about 50 mg Compound A free base. In some embodiments, the subject weighs from about 10 kg to about 20 kg and the second dose is in an amount equivalent to about 75 mg Compound A free base. In some embodiments, the subject weighs from about 10 kg to about 20 kg, the first dose is in an amount equivalent to about 25 mg Compound A free base, and the second dose is in an amount equivalent to about 50 mg Compound A free base. In some embodiments, the subject weighs from about 10 kg to about 20 kg, the first dose is in an amount equivalent to about 0 mg Compound A free base, and the second dose is in an amount equivalent to about 50 mg Compound A free base. In some embodiments, the subject weighs from about 10 kg to about 20 kg, the first dose is in an amount equivalent to about 0 mg Compound A free base, and the second dose is in an amount equivalent to about 75 mg Compound A free base.

In some embodiments, prior to the administering of a first dose equivalent to about 0 mg Compound A free base q.A.M. and a second dose equivalent to about 50 mg Compound A free base q.P.M., the subject that weighs from about 10 kg to about 20 kg demonstrated adequate disease control and was being administered a first dose equivalent to about 25 mg Compound A free base q.A.M. and a second dose equivalent to about 50 mg Compound A free base q.P.M. In some embodiments, prior to the administering of a first dose equivalent to about 0 mg Compound A free base q.A.M. and a second dose equivalent to about 50 mg Compound A free base q.P.M., the subject that weighs from about 10 kg to about 20 kg demonstrated adequate disease control and was being administered a first dose equivalent to about 25 mg Compound A free base q.A.M. and a second dose equivalent to about 50 mg Compound A free base q.P.M. and a glucocorticoid at a target dose.

In some embodiments, prior to the administering of a first dose equivalent to about 0 mg Compound A free base q.A.M. and a second dose equivalent to about 75 mg Compound A free base q.P.M., the subject that weighs from about 10 kg to about 20 kg demonstrated adequate disease control and was being administered a first dose equivalent to about 25 mg Compound A free base q.A.M. and a second dose equivalent to about 50 mg Compound A free base q.P.M. In some embodiments, prior to the administering of a first dose equivalent to about 0 mg Compound A free base q.A.M. and a second dose equivalent to about 75 mg Compound A free base q.P.M., the subject that weighs from about 10 kg to about 20 kg demonstrated adequate disease control and was being administered a first dose equivalent to about 25 mg Compound A free base q.A.M. and a second dose equivalent to about 50 mg Compound A free base q.P.M. and a glucocorticoid at a target dose.

In some embodiments, the subject demonstrated adequate disease control if their androstenedione levels are ≤120% of the subject's baseline or ≤upper limit of normal (ULN) for age and sex of the subject.

In some embodiments, the subject demonstrated adequate disease control in the opinion of the investigator.

In some embodiments, the subject weighs from about 10 kg to about 20 kg and, prior to administration of Compound A, or a pharmaceutically acceptable salt thereof, in two doses consisting of a first dose and a second dose wherein the second dose is greater than the first dose, the subject had been administered Compound A, or a pharmaceutically acceptable salt thereof, as a twice daily dose, each dose having an equivalent amount of Compound A free base for a first period of time. In some embodiments, each dose is in an amount equivalent to about 25 mg of Compound A free base.

In some embodiments, the method further comprises adjusting for a missed q.A.M. dose. In some embodiments, the method further comprises adjusting for a missed q.P.M. dose. In some embodiments, in the event of a missed dose, the amount of the missed dose is administered as soon as possible, even if it is soon before the next scheduled dose, and then the regular dosing schedule is resumed.

Further provided is a method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

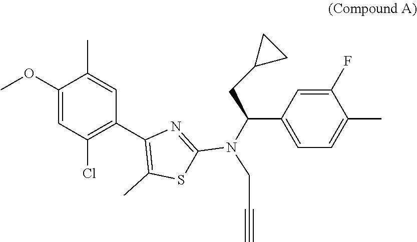

as an adjunct to glucocorticoid therapy in a pediatric subject having congenital adrenal hyperplasia (CAH), the method comprising: administering to the subject a first amount of Compound A, or a pharmaceutically acceptable salt thereof, for a first period of time, and subsequently administering to the subject a second amount of Compound A, or a pharmaceutically acceptable salt thereof, for a second period of time, wherein the second amount is administered in two doses consisting of a first dose and a second dose wherein the second dose is greater than the first dose. In some embodiments, the method further comprises subsequently administering to the subject a third amount of Compound A, or a pharmaceutically acceptable salt thereof, for a third period of time, wherein the third amount is administered in a q.P.M. dose.

Also provided is a method of treating congenital adrenal hyperplasia (CAH) in a pediatric subject already being treated with glucocorticoid therapy, the method comprising: administering to the subject a first amount of 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

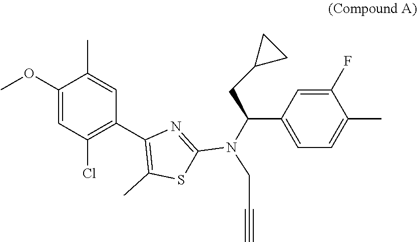

for a first period of time, and subsequently administering to the subject a second amount of Compound A, or a pharmaceutically acceptable salt thereof, for a second period of time, wherein the second amount is administered in two doses consisting of a first dose and a second dose wherein the second dose is greater than the first dose. In some embodiments, the method further comprises subsequently administering to the subject a third amount of Compound A, or a pharmaceutically acceptable salt thereof, for a third period of time, wherein the third amount is administered in a q.P.M. dose.

Further provided is a compound which is 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine:

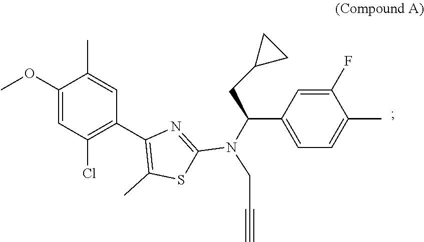

or a pharmaceutically acceptable salt thereof;
for use in a method of treating congenital adrenal hyperplasia (CAH) in a pediatric subject already being treated with glucocorticoid therapy, the method comprising:
   (1) administering to the subject a first amount of Compound A, or a pharmaceutically acceptable salt thereof, for a first period of time, and
   (2) subsequently administering to the subject a second amount of Compound A, or a pharmaceutically acceptable salt thereof, for a second period of time, wherein the second amount is administered in two doses consisting of a first dose and a second dose wherein the second dose is greater than the first dose. In some embodiments, the method further comprises: (3) subsequently administering to the subject a third amount of Compound A, or a pharmaceutically acceptable salt thereof, for a third period of time, wherein the third amount is administered in a q.P.M. dose.

Further provided is the use of a compound which is 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine:

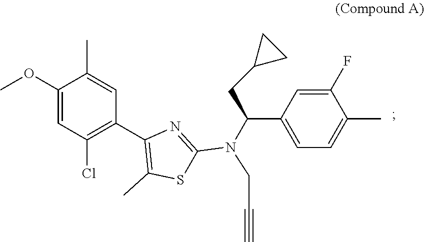

or a pharmaceutically acceptable salt thereof;
in the manufacture of a medicament for treating congenital adrenal hyperplasia (CAH) in a pediatric subject already being treated with glucocorticoid therapy, by a method comprising the steps:
   (1) administering to the subject a first amount of Compound A, or a pharmaceutically acceptable salt thereof, for a first period of time, and
   (2) subsequently administering to the subject a second amount of Compound A, or a pharmaceutically acceptable salt thereof, for a second period of time, wherein the second amount is administered in two doses consisting of a first dose and a second dose wherein the second dose is greater than the first dose. In some embodiments, the method further comprises: (3) subsequently administering to the subject a third amount of Compound A, or a pharmaceutically acceptable salt thereof, for a third period of time, wherein the third amount is administered in a q.P.M. dose.

In some embodiments, administering Compound A, or a pharmaceutically acceptable salt thereof, refers to administration of Compound A, or a pharmaceutically acceptable salt thereof, with food. In some embodiments, the administration of Compound A, or a pharmaceutically acceptable salt thereof, with food exhibits a positive food effect. In some embodiments, an increased, or positive, food effect is observed when Compound A, or a pharmaceutically acceptable salt thereof is administered to a subject in a fed state. In some embodiments, administration of Compound A, or a pharmaceutically acceptable salt thereof, results in an increased, or positive, food effect, whereby an increased Cₘₐₓ and/or AUC are observed when administered orally in the fed state as compared to the fasting state.

In some embodiments, administering Compound A, or a pharmaceutically acceptable salt thereof, refers to administration of Compound A, or a pharmaceutically acceptable salt thereof, with the subject's breakfast and evening meal.

In some embodiments, the subject weighs greater than or equal to about 55 kg.

In some embodiments, the first amount is equivalent to about 100 mg Compound A free base b.i.d. In some embodiments, the first amount is equivalent to about 100 mg Compound A free base q.A.M. and about 100 mg Compound A free base q.P.M.

In some embodiments, the second amount is equivalent to about 100 mg of Compound A free base q.A.M. and about 200 mg Compound A free base q.P.M. In some embodiments, the third amount is equivalent to about 200 mg Compound A free base q.P.M. In some embodiments, the third amount is equivalent to about 300 mg Compound A free base q.P.M.

In some embodiments, the first period of time is from about 1 week to about 1 year.

In some embodiments, the second period of time is at least about 1 week.

In some embodiments, the third period of time is at least about 1 week.

In some embodiments, the subject weighs from about 20 kg to about 55 kg.

In some embodiments, the first amount is equivalent to about 50 mg Compound A free base b.i.d. In some embodiments, the first amount is equivalent to about 50 mg Compound A free base q.A.M. and about 50 mg Compound A free base q.P.M.

In some embodiments, the second amount is equivalent to about 50 mg of Compound A free base q.A.M. and about 100 mg Compound A free base q.P.M.

In some embodiments, the third amount is equivalent to about 100 mg Compound A free base q.P.M. In some embodiments, the third amount is equivalent to about 150 mg Compound A free base q.P.M.

In some embodiments, the first period of time is from about 1 week to about 1 year.

In some embodiments, the second period of time is at least about 1 week.

In some embodiments, the third period of time is at least about 1 week.

In some embodiments, the subject weighs from about 10 kg to about 20 kg.

In some embodiments, the first amount is equivalent to about 25 mg Compound A free base b.i.d. In some embodiments, the first amount is equivalent to about 25 mg Compound A free base q.A.M. and about 25 mg Compound A free base q.P.M.

In some embodiments, the second amount is equivalent to about 25 mg of Compound A free base q.A.M. and about 50 mg Compound A free base q.P.M.

In some embodiments, the third amount is equivalent to about 50 mg Compound A free base q.P.M. In some embodiments, the third amount is equivalent to about 75 mg Compound A free base q.P.M.

In some embodiments, the first period of time is from about 1 week to about 1 year.

In some embodiments, the second period of time is at least about 1 week.

In some embodiments, the third period of time is at least about 1 week.

In some embodiments, the first period of time is between 1 week and 1 year. In some embodiments, the first period of time is from about 1 week to about 1 year. In some embodiments, the first period of time is at least 6 months. In some embodiments, the first period of time is less than 6 months. In some embodiments, the first period of time is more than 6 months. In some embodiments, the first period of time is about 6 months. In some embodiments, the first period of time is at least one year. In some embodiments, the first period of time is greater than one year. In some embodiments, the first period of time is about one year.

In some embodiments, the second period of time is at least about 1 week. In some embodiments, the second period of time is between 1 week and 1 year. In some embodiments, the second period of time is from about 1 week to about 1 year. In some embodiments, the second period of time is at least 6 months. In some embodiments, the second period of time is less than 6 months. In some embodiments, the second period of time is more than 6 months. In some embodiments, the second period of time is about 6 months. In some embodiments, the second period of time is at least one year. In some embodiments, the second period of time is greater than one year. In some embodiments, the second period of time is less than one year. In some embodiments, the second period of time is about one year. In some embodiments, the third period of time is at least about 1 week. In some embodiments, the third period of time is between 1 week and 1 year. In some embodiments, the third period of time is from about 1 week to about 1 year. In some embodiments, the third period of time is at least 6 months. In some embodiments, the third period of time is less than 6 months. In some embodiments, the third period of time is more than 6 months. In some embodiments, the third period of time is about 6 months. In some embodiments, the third period of time is at least one year. In some embodiments, the third period of time is greater than one year. In some embodiments, the third period of time is less than one year. In some embodiments, the third period of time is about one year.

In some embodiments, the second period of time begins immediately after the conclusion of the first period of time. In some embodiments, the second period of time begins within 1 day after the conclusion of the first period of time. In some embodiments, the second period of time begins within 1 week after the conclusion of the first period of time. In some embodiments, the second period of time begins within 1 month after the conclusion of the first period of time. In some embodiments, the second period of time begins within 6 months after the conclusion of the first period of time. In some embodiments, the second period of time begins about 1 day after the conclusion of the first period of time. In some embodiments, the second period of time begins about 1 week after the conclusion of the first period of time. In some embodiments, the second period of time begins about 1 month after the conclusion of the first period of time. In some embodiments, the second period of time begins about 6 months after the conclusion of the first period of time.

In some embodiments, the third period of time begins immediately after the conclusion of the second period of time. In some embodiments, the third period of time begins within 1 day after the conclusion of the second period of time. In some embodiments, the third period of time begins within 1 week after the conclusion of the second period of time. In some embodiments, the third period of time begins within 1 month after the conclusion of the second period of time. In some embodiments, the third period of time begins within 6 months after the conclusion of the second period of time. In some embodiments, the third period of time begins about 1 day after the conclusion of the second period of time. In some embodiments, the third period of time begins about 1 week after the conclusion of the second period of time. In some embodiments, the third period of time begins about 1 month after the conclusion of the second period of time. In some embodiments, the third period of time begins about 6 months after the conclusion of the second period of time.

In some embodiments, the method further comprises adjusting for a missed q.A.M. dose. In some embodiments, the method further comprises adjusting for a missed q.P.M. dose. In some embodiments, in the event of a missed dose, the amount of the missed dose is administered as soon as possible, even if it is soon before the next scheduled dose, and then the regular dosing schedule is resumed.

In some embodiments, the glucocorticoid therapy is a daily dose of a glucocorticoid administered to the subject.

In some embodiments, the daily dose of the glucocorticoid is administered once daily or through multiple administrations. In some embodiments, the daily dose of the glucocorticoid is administered once daily. In some embodiments, the daily dose of the glucocorticoid is administered through multiple administrations.

In some embodiments, the glucocorticoid is selected from hydrocortisone, prednisone, methylprednisolone, prednisolone, dexamethasone, and fludrocortisone, or a combination thereof. In some embodiments, the glucocorticoid is selected from hydrocortisone, prednisone, methylprednisolone, prednisolone, and dexamethasone, or a combination thereof. In some embodiments, the glucocorticoid is hydrocortisone. In some embodiments, the glucocorticoid is prednisone. In some embodiments, the glucocorticoid is methylprednisolone. In some embodiments, the glucocorticoid is prednisolone. In some embodiments, the glucocorticoid is dexamethasone. In some embodiments, the glucocorticoid is fludrocortisone.

In some embodiments, prior to the administering of Compound A, or a pharmaceutically acceptable salt thereof, the subject was receiving a glucocorticoid at a target dose.

In some embodiments, prior to the administering of the second amount of Compound A, or a pharmaceutically acceptable salt thereof, the subject was receiving a glucocorticoid at a target dose. In some embodiments, prior to the administering of the second amount of Compound A, or a pharmaceutically acceptable salt thereof, for the second period of time, the subject was receiving a glucocorticoid at a target dose.

In some embodiments, prior to the administering of the third amount of Compound A, or a pharmaceutically acceptable salt thereof, the subject was receiving a glucocorticoid at a target dose. In some embodiments, prior to the administering of the third amount of Compound A, or a pharmaceutically acceptable salt thereof, for the third period of time, the subject was receiving a glucocorticoid at a target dose.

In some embodiments, the target dose of glucocorticoid treatment is ≤11 mg/m²/day hydrocortisone equivalents.

In some embodiments, the target dose of glucocorticoid treatment is about 8 mg/m²/day to about 10 mg/m²/day hydrocortisone equivalents.

In some embodiments, the target dose of glucocorticoid treatment is about 8 mg/m²/day hydrocortisone equivalents.

In some embodiments, the glucocorticoid is hydrocortisone. In some embodiments, the glucocorticoid is methylprednisolone using a hydrocortisone dose equivalent conversion factor of about 4×. In some embodiments, the glucocorticoid is prednisolone using a hydrocortisone dose equivalent conversion factor of about 4×. In some embodiments, the glucocorticoid is prednisone using a hydrocortisone dose equivalent conversion factor of about 4×. In some embodiments, the glucocorticoid is methylprednisolone using a hydrocortisone dose equivalent conversion factor of about 5×. In some embodiments, the glucocorticoid is prednisolone using a hydrocortisone dose equivalent conversion factor of about 5×. In some embodiments, the glucocorticoid is prednisone using a hydrocortisone dose equivalent conversion factor of about 5×. In some embodiments, the glucocorticoid is dexamethasone using a hydrocortisone dose equivalent conversion factor from about 26× to about 80×. In some embodiments, the glucocorticoid is dexamethasone using a hydrocortisone dose equivalent conversion factor of about 60×. In some embodiments, the glucocorticoid is dexamethasone using a hydrocortisone dose equivalent conversion factor of about 80×.

In some embodiments, prior to the administering of Compound A, or a pharmaceutically acceptable salt thereof, the subject demonstrated adequate disease control.

In some embodiments, prior to the administering of the second amount of Compound A, or a pharmaceutically acceptable salt thereof, the subject had inadequate efficacy. In some embodiments, prior to the administering of the second amount of Compound A, or a pharmaceutically acceptable salt thereof, for the second period of time, the subject had inadequate efficacy.

In some embodiments, prior to the administering of the third amount of Compound A, or a pharmaceutically acceptable salt thereof, the subject demonstrated adequate disease control.

In some embodiments, prior to the administering of Compound A, or a pharmaceutically acceptable salt thereof, the subject demonstrated adequate disease control and was being administered a glucocorticoid at the target dose.

In some embodiments, prior to the administering of the second amount of Compound A, or a pharmaceutically acceptable salt thereof, the subject had inadequate efficacy and was being administered a glucocorticoid at the target dose. In some embodiments, prior to the administering of the second amount of Compound A, or a pharmaceutically acceptable salt thereof, for the second period of time, the subject had inadequate efficacy and was being administered a glucocorticoid at the target dose.

In some embodiments, prior to the administering of the third amount of Compound A, or a pharmaceutically acceptable salt thereof, the subject demonstrated adequate disease control and was being administered a glucocorticoid at the target dose.

In some embodiments, the subject demonstrated adequate disease control if their androstenedione levels are ≤120% of the subject's baseline or ≤upper limit of normal (ULN) for age and sex of the subject.

In some embodiments, the subject had adequate efficacy if their androstenedione levels are ≤120% of the subject's baseline or ≤upper limit of normal (ULN) for age and sex of the subject.

In some embodiments, the subject demonstrated adequate disease control in the opinion of the investigator. In some embodiments, the subject had adequate efficacy in the opinion of the investigator. In some embodiments, the subject demonstrated adequate disease control in the opinion of the prescriber or health care provider. In some embodiments, the subject had adequate efficacy in the opinion of the prescriber or health care provider.

In some embodiments, the method further comprises monitoring the subject for adequate disease control, wherein if the disease control is inadequate and the glucocorticoid therapy is at or above the target dose of the subject, then the amount of Compound A, or a pharmaceutically acceptable salt thereof, is adjusted. In some embodiments, the use further comprises monitoring the subject for adequate disease control, wherein if the disease control is inadequate and the glucocorticoid therapy is at or above the target dose of the subject, then the amount of Compound A, or a pharmaceutically acceptable salt thereof, is adjusted.

In some embodiments, the subject demonstrated inadequate disease control if their androstenedione levels are >120% of the subject's baseline or >upper limit of normal (ULN) for age and sex of the subject.

In some embodiments, the subject had inadequate efficacy if their androstenedione levels are >120% of the subject's baseline or >upper limit of normal (ULN) for age and sex of the subject.

In some embodiments, the subject demonstrated inadequate disease control in the opinion of the investigator. In some embodiments, the subject had inadequate efficacy in the opinion of the investigator. In some embodiments, the subject demonstrated inadequate disease control in the opinion of the prescriber or health care provider. In some embodiments, the subject had inadequate efficacy in the opinion of the prescriber or health care provider.

In some embodiments, Compound A, or a pharmaceutically acceptable salt thereof, is a pharmaceutically acceptable salt of Compound A. In some embodiments, Compound A, or a pharmaceutically acceptable salt thereof, is the free base of Compound A. In some embodiments, the free base of Compound A is crystalline. In some embodiments, the free base of Compound A is a crystalline form as described in International Application Publication No. WO 2021/111179.

In some embodiments, Compound A, or a pharmaceutically acceptable salt thereof, is administered in a pharmaceutical composition further comprising one or more pharmaceutically acceptable excipients. In some embodiments, the methods and uses described herein comprise administering a pharmaceutical composition that is the formulation described in Example 2 herein. In some embodiments, the methods and uses described herein comprise administering a pharmaceutical composition that is a formulation described in WO 2020/115555.

In some embodiments, the pharmaceutical composition is in an oral solution dosage form comprising:
(a) Compound A, or a pharmaceutically acceptable salt thereof;
(b) one or more of a sweetener, an anti-oxidant, and a flavor; and
(c) a liquid vehicle.

In some embodiments, the pharmaceutical composition comprises about 1 w/v % to about 50 w/v % of the compound of Compound A, or a pharmaceutically acceptable salt thereof, based on the weight of the free base. In some embodiments, the pharmaceutical composition comprises about 1 w/v % to about 10 w/v % of the compound of Compound A, or a pharmaceutically acceptable salt thereof, based on the weight of the free base. In some embodiments, the pharmaceutical composition comprises about 5 w/v % of the compound of Compound A, or a pharmaceutically acceptable salt thereof, based on the weight of the free base. In some embodiments, the pharmaceutical composition comprises about 1, 2, 3, 4, 5, 6, 7, 8, 9, or 10 w/v % of the compound of Compound A, or a pharmaceutically acceptable salt thereof, based on the weight of the free base, or within a range of any of the preceding values.

In some embodiments, the pharmaceutical composition comprises a sweetener. A sweetener is a formulation component added to improve taste. In some embodiments, the pharmaceutical composition comprises about 0.01 w/v % to about 1.5 w/v % of the sweetener. In some embodiments, the pharmaceutical composition comprises about 0.1 w/v % to about 0.5 w/v % of the sweetener. In some embodiments, the pharmaceutical composition comprises about 0.15 w/v % of the sweetener. In some embodiments, the pharmaceutical composition comprises about 0.1, 0.2, 0.3, 0.4, or 0.5 w/v % of the sweetener, or within a range of any of the preceding values.

In some embodiments, the sweetener is selected from saccharin, sucrose, sucralose, aspartame, dextrose, fructose, maltitol, mannitol, sorbitol, and avantame. In some embodiments, the sweetener is saccharin.

In some embodiments, the pharmaceutical composition comprises an anti-oxidant. An anti-oxidant is a formulation component included to improve stability by preventing oxidation. In some embodiments, the pharmaceutical composition comprises about 0.01 w/v % to about 1.5 w/v % of the anti-oxidant. In some embodiments, the pharmaceutical composition comprises about 0.1 w/v % to about 0.5 w/v % of the anti-oxidant. In some embodiments, the pharmaceutical composition comprises about 0.17 w/v % of the anti-oxidant. In some embodiments, the pharmaceutical composition comprises about 0.1, 0.2, 0.3, 0.4, or 0.5 w/v % of the anti-oxidant, or within a range of any of the preceding values.

In some embodiments, the anti-oxidant is selected from butylated hydroxytoluene, vitamin E TPGS, butylated hydroxyanisole, ascorbic acid, lecithin, tert-butylhydroquinone, and citric acid. In some embodiments, the anti-oxidant is butylated hydroxytoluene.

In some embodiments, the pharmaceutical composition comprises a flavor. A flavor is a formulation component added to mask taste through aromatics. In some embodiments, the pharmaceutical composition comprises about 0.01 w/v % to about 0.5 w/v % of the flavor. In some embodiments, the pharmaceutical composition comprises about 0.05 w/v % to about 0.2 w/v % of the flavor. In some embodiments, the pharmaceutical composition comprises about 0.10 w/v % of the flavor. In some embodiments, the pharmaceutical composition comprises about 0.05, 0.06, 0.07, 0.08, 0.09, 0.10, 0.11, 0.12, 0.13, 0.14, 0.15, 0.16, 0.17, 0.18, 0.19 or 0.2 w/v % of the flavor, or within a range of any of the preceding values.

In some embodiments, the flavor is selected from FONA orange flavor, FONA Juicy Flavor, FONA Grape Flavor, Firmenich SA Lemon Flavor, Firmenich Tetrarome Orange Flavor, IFF Cherry Flavor, and IFF Grape Flavor. In some embodiments, the flavor is FONA orange flavor. In some embodiments, the flavor is orange flavor.

A liquid vehicle is a solvent capable of dissolving or partially dissolving the compound of Compound A, or a pharmaceutically acceptable salt thereof, for the purposes of delivery as an oral dosing solution. In some embodiments, the pharmaceutical composition comprises about 50 w/v % to about 99.9 w/v % of the liquid vehicle. In some embodiments, the pharmaceutical composition comprises about 90 w/v % to about 99 w/v % of the liquid vehicle. In some embodiments, the pharmaceutical composition comprises about 92 w/v % to about 97 w/v % of the liquid vehicle. In some embodiments, the pharmaceutical composition comprises about 94.6 w/v % of the liquid vehicle. In some embodiments, the pharmaceutical composition comprises about 90, 91, 92, 93, 94, 95, 96, 97, 98 or 99 w/v % of the liquid vehicle, or within a range of any of the preceding values.

In some embodiments, the liquid vehicle is selected from medium-chain triglycerides, propylene glycol dicaprylate/dicaprate, glycerin, propylene glycol, polyethylene glycol, olive oil, soybean oil, corn oil, and transcutol. In some embodiments, the liquid vehicle is medium-chain triglycerides. In some embodiments, the medium-chain triglycerides is Labrafac™ Lipophile WL1349.

In some embodiments, the pharmaceutical composition further comprises a surfactant. A surfactant is a formulation component added to improve solubility or emulsion properties. In some embodiments, the pharmaceutical composition comprises about 1 w/v % to about 50 w/v % of the surfactant. In some embodiments, the pharmaceutical composition comprises about 10 w/v % to about 30 w/v % of the surfactant. In some embodiments, the pharmaceutical composition comprises about 20 w/v % of the surfactant. In some embodiments, the pharmaceutical composition comprises about 10, 11, 12, 13, 14, 15, 16, 17, 18, 19, 20, 21, 22, 23, 24, 25, 26, 27, 28, 29 or 30 w/v % of the surfactant, or within a range of any of the preceding values.

In some embodiments, the surfactant is selected from oleoyl polyoxyl-6 glycerides, linoleoyl polyoxyl-6 glycerides, Polysorbate 80, Polysorbate 20, vitamin E polyethylene glycol succinate, Gelucire, lauroyl polyoxyl-32 glycerides, sodium lauryl sulfate, Poloxamer, corn oil PEG-6 esters, and hydrogenated palm/palm kernel oil PEG-6 esters. In some embodiments, the surfactant is oleoyl polyoxyl-6 glycerides. In some embodiments, the oleoyl polyoxyl-6 glycerides is LABRAFIL™ M 1944 CS.

In some embodiments, the pharmaceutical composition comprises about 50 w/v % to about 90 w/v % of the liquid vehicle. In some embodiments, the pharmaceutical composition comprises about 70 w/v % to about 80 w/v % of the liquid vehicle. In some embodiments, the pharmaceutical composition comprises about 75 w/v % of the liquid vehicle. In some embodiments, the pharmaceutical composition comprises about 74.6 w/v % of the liquid vehicle. In some embodiments, the pharmaceutical composition comprises about 70, 71, 72, 73, 74, 75, 76, 77, 78, 79, or 80 w/v % of the liquid vehicle, or within a range of any of the preceding values.

In some embodiments, the liquid vehicle is selected from medium-chain triglycerides, propylene glycol dicaprylate/dicaprate, glycerin, propylene glycol, polyethylene glycol, olive oil, soybean oil, corn oil, and transcutol. In some embodiments, the liquid vehicle is medium-chain triglycerides. In some embodiments, the medium-chain triglycerides is Labrafac™ Lipophile WL1349.

In some embodiments, the pharmaceutical composition comprises:
(a) Compound A, or a pharmaceutically acceptable salt thereof;
(b) a sweetener;
(c) an anti-oxidant;
(d) a flavor; and
(e) a liquid vehicle.

In some embodiments, the pharmaceutical composition further comprises a surfactant.

In some embodiments, the pharmaceutical composition comprises:
(a) about 4 w/v % to about 6 w/v % of Compound A, or a pharmaceutically acceptable salt thereof, based on the weight of the free base;
(b) about 0.1 w/v % to about 0.2 w/v % of a sweetener;
(c) about 0.1 w/v % to about 0.2 w/v % of an anti-oxidant;
(d) about 0.05 w/v % to about 0.2 w/v % of a flavor; and
(e) about 92 w/v % to about 97 w/v % of a liquid vehicle.

In some embodiments, the pharmaceutical composition comprises:
(a) about 5 w/v % of Compound A, or a pharmaceutically acceptable salt thereof, based on the weight of the free base;
(b) about 0.15 w/v % of a sweetener;
(c) about 0.17 w/v % of an anti-oxidant;
(d) about 0.1 w/v % of a flavor; and
(e) about 94.6 w/v % of a liquid vehicle.

In some embodiments, the pharmaceutical composition comprises:
(a) about 4 w/v % to about 6 w/v % of Compound A, or a pharmaceutically acceptable salt thereof, based on the weight of the free base;
(b) about 0.1 w/v % to about 0.2 w/v % of a sweetener;
(c) about 0.1 w/v % to about 0.2 w/v % of an anti-oxidant;
(d) about 0.05 w/v % to about 0.2 w/v % of a flavor;
(e) about 15 w/v % to about 25 w/v % of a surfactant; and
(f) about 70 w/v % to about 80 w/v % of a liquid vehicle.

In some embodiments, the pharmaceutical composition comprises:
(a) about 5 w/v % of Compound A, or a pharmaceutically acceptable salt thereof, based on the weight of the free base;
(b) about 0.15 w/v % of a sweetener;
(c) about 0.17 w/v % of an anti-oxidant;
(d) about 0.1 w/v % of a flavor;
(e) about 20 w/v % of a surfactant; and
(f) about 75 w/v % of a liquid vehicle.

In some embodiments, the pharmaceutical composition comprises:
(a) Compound A, or a pharmaceutically acceptable salt thereof;
(b) saccharin;
(c) butylated hydroxytoluene;
(d) FONA orange flavor; and
(e) medium-chain triglycerides.

In some embodiments, the pharmaceutical composition further comprises oleoyl polyoxyl-6 glycerides.

In some embodiments, the pharmaceutical composition comprises:
(a) about 4 w/v % to about 6 w/v % of Compound A, or a pharmaceutically acceptable salt thereof, based on the weight of the free base;
(b) about 0.1 w/v % to about 0.2 w/v % of saccharin;
(c) about 0.1 w/v % to about 0.2 w/v % of butylated hydroxytoluene;
(d) about 0.05 w/v % to about 0.2 w/v % of FONA orange flavor; and
(e) about 92 w/v % to about 97 w/v % of medium-chain triglycerides.

In some embodiments, the pharmaceutical composition comprises:
(a) about 5 w/v % of Compound A, or a pharmaceutically acceptable salt thereof, based on the weight of the free base;
(b) about 0.15 w/v % of saccharin;
(c) about 0.17 w/v % of butylated hydroxytoluene;
(d) about 0.1 w/v % of FONA orange flavor; and
(e) about 94.6 w/v % of medium-chain triglycerides.

In some embodiments, the pharmaceutical composition comprises:
(a) about 4 w/v % to about 6 w/v % of Compound A, or a pharmaceutically acceptable salt thereof, based on the weight of the free base;
(b) about 0.1 w/v % to about 0.2 w/v % of saccharin;
(c) about 0.1 w/v % to about 0.2 w/v % of butylated hydroxytoluene;
(d) about 0.05 w/v % to about 0.2 w/v % of FONA orange flavor;
(e) about 15 w/v % to about 25 w/v % of oleoyl polyoxyl-6 glycerides; and
(f) about 70 w/v % to about 80 w/v % of medium-chain triglycerides.

In some embodiments, the pharmaceutical composition comprises:
(a) about 5 w/v % of Compound A, or a pharmaceutically acceptable salt thereof, based on the weight of the free base;
(b) about 0.15 w/v % of saccharin;
(c) about 0.17 w/v % of butylated hydroxytoluene;
(d) about 0.1 w/v % of FONA orange flavor;
(e) about 20 w/v % of oleoyl polyoxyl-6 glycerides; and
(f) about 75 w/v % of medium-chain triglycerides.

In some embodiments, the pharmaceutical composition comprises the compound of Compound A as a free base. In some embodiments, the pharmaceutical composition comprises the compound of Compound A as the free base of Compound A.

In some embodiments, the pharmaceutical composition is formulated in unit dosage form, wherein the compound of Compound A, or a pharmaceutically acceptable salt thereof, is present in an amount of about 5 mg/mL to about 200 mg/mL, based on the weight of the free base. In some embodiments, the compound of Compound A, or a pharmaceutically acceptable salt thereof, is present in the unit dosage form in an amount of about 75 mg/mL to about 150 mg/mL, based on the weight of the free base. In some embodiments, the compound of Compound A, or a pharmaceutically acceptable salt thereof, is present in the unit dosage form in an amount of about 50 mg/mL, based on the weight of the free base. In some embodiments, the compound of Compound A, or a pharmaceutically acceptable salt thereof, is present in the unit dosage form in an amount of about 100 mg/mL, based on the weight of the free base.

In some embodiments, the liquid pharmaceutical composition has a viscosity between about 1 to about 50 centipoise at about 25° C.

In some embodiments, the CAH is classical Congenital Adrenal Hyperplasia.

In some embodiments, the CAH is non-classical Congenital Adrenal Hyperplasia.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### Example 1: Phase 3 Compound A Pediatric Studies

This is a Phase 3, randomized, double-blind, placebo-controlled study to evaluate the efficacy, safety, and tolerability of Compound A versus placebo administered twice daily (b.i.d.) with breakfast and evening meals for 28 weeks in approximately 81 pediatric subjects with classic congenital adrenal hyperplasia (CAH) due to 21-hydroxylase deficiency. Eligible subjects will be randomly assigned in a 2:1 ratio (active:placebo) to either Compound A (25 mg b.i.d. via oral solution for subjects 10 to <20 kg, 50 mg b.i.d. via oral solution for subjects 20 to <55 kg, or 100 mg b.i.d. via oral capsules for subjects ≥55 kg) or matching placebo (oral solution placebo for subjects <55 kg and oral capsule placebo for subjects ≥55 kg). Dose assignment from Day 1 to Week 28 will be based on the subject's weight at Day 1. After the 28-week placebo-controlled treatment period, there will be a 24-week, open-label treatment period, during which all subjects will receive Compound A at doses based on their Week 28 body weight.

At Month 12 (Week 52), subjects will have the option to participate in an open-label extension (OLE). During the OLE, subjects will continue to receive Compound A at their weight-based dose, unless the subject has inadequate efficacy (in the opinion of the investigator), in which case their Compound A dose can be increased by doubling the evening dose. At Month 12, subjects can also elect to switch the Compound A formulation (oral solution or capsule) based on preference.

### Screening Period (Weeks −4 Up to Day −1)

Subjects will undergo screening for up to 4 weeks (Week −4 to Day −1) to determine eligibility. Prior to the blood sample collection during screening, subjects will be asked to take their evening glucocorticoid dose the night before prior to 2100 hours and hold their morning dose of glucocorticoid and fludrocortisone (if applicable).

### Randomized, Double-Blind, Placebo-Controlled Treatment Period (Day 1 Up to Week 28)

For visits at which blood samples are collected, subjects should take their evening glucocorticoid dose (if applicable) the night before prior to 2100 hours and hold their morning dose of glucocorticoid, fludrocortisone (if applicable), and study drug (after Day 1) until after predose blood sample collection, with dosing to occur between approximately 0800 and 0830 hours with breakfast. For blood tests obtained at the study site, subjects should bring their morning dose of glucocorticoid, fludrocortisone (if applicable), and study drug (after Day 1) with them to the study site to take at the study site (with dosing to occur between approximately 0800 and 0830 hours with breakfast). Except for the screening visit, subjects should be fasting after midnight the night before until after the predose blood sample collection(s) but should be encouraged to drink water to avoid any hypovolemic status. Subjects <6 years of age for whom the investigator deems that fasting until 0800 hours is not feasible can have a snack(s) if needed but should wait to have breakfast with morning dosing.

### Glucocorticoid-Stable Period

On Day 1, subjects who are ≥6 years of age with a body weight ≥20 kg should arrive at the study site for the serial blood sampling procedure and dosing, with dosing to occur between approximately 0800 and 0830 hours. Blood samples will be obtained serially over approximately 6 hours, with 2 baseline samples obtained approximately 15 minutes before the morning glucocorticoid dose and again immediately prior to the morning glucocorticoid dose administration (with dosing to occur between approximately 0800 and 0830 hours) and at 2, 3, 4, and 6 hours after the morning glucocorticoid dose, for a total of 6 blood sampling time points. Subjects who are <6 years of age or with a body weight <20 kg will have a blood sample collection prior to their morning glucocorticoid dose only (with dosing to occur between approximately 0800 and 0830 hours). For all subjects, salivary samples for adrenal androgens and precursors will be collected in the early morning (0600 hours), at approximately 0800 hours prior to the morning glucocorticoid dose, at approximately 3 and 6 hours after the morning glucocorticoid dose, and in the evening (within 30 minutes prior to the evening dose of glucocorticoid; if subject is not on an evening glucocorticoid dose, then prior to bedtime). The salivary samples at approximately 0800 hours prior to dosing and 3 and 6 hours after dosing should be collected at approximately the same time as any corresponding blood samples (as applicable). Any midday glucocorticoid dose that the subject is receiving should be taken after the 6-hour postdose blood and salivary samples are collected. During the study, the exact timing of the blood and saliva samples, and the morning glucocorticoid, mineralocorticoid, and/or study drug dose (and breakfast), may be adjusted by ±1 hour to accommodate site and subject schedules if needed as long as this is done consistently per subject and the relative times between the sample(s) and the dose(s) are maintained.

Subjects will be randomized on Day 1 in a 2:1 ratio (active:placebo). Randomization will be stratified by pubertal stage (Tanner breast or genital stage 1 or 2 versus 3, 4 or 5) and sex. From the evening of the Day 1 visit (after all Day 1 assessments have been performed) until the morning of the Week 28 visit, all subjects will receive blinded study drug based on their Day 1 weight (25 mg b.i.d. via oral solution for subjects 10 to <20 kg, 50 mg b.i.d. via oral solution for subjects 20 to <55 kg, or 100 mg b.i.d. via oral capsules for subjects ≥55 kg). Study drug will be administered b.i.d. with the subject's breakfast and evening meals (each dose separated by approximately 12 hours).

From Day 1 until Week 4, subjects should maintain a stable glucocorticoid regimen to the extent possible, except for stress dosing if needed for illness or other significant physical stress, in order to assess the direct effect of study drug on adrenal androgens and precursors. Stress dosing of glucocorticoid (at any time during the study) can be based on guidance provided by the investigator, the subject's treating physician, or guidelines provided in the protocol.

### Glucocorticoid-Adjustment Period

At the Week 4 visit, subjects ≥6 years of age with a body weight ≥20 kg should arrive at the study site for the serial blood sampling procedure and dosing, with dosing to occur between approximately 0800 and 0830 hours with breakfast. Blood samples will be obtained serially over approximately 6 hours, with 2 baseline samples (obtained approximately 15 minutes before and again immediately prior to the morning glucocorticoid and study drug dose, with dosing to occur between approximately 0800 and 0830 hour). Blood samples will also be obtained at 2, 3, 4, and 6 hours after dosing. Pharmacokinetic (PK) blood samples will be obtained at the same time points with the exception of only 1 required predose sample. Subjects who are <6 years of age or with a body weight <20 kg will have blood sample collection prior to the morning glucocorticoid and study drug dose only (with dosing to occur between approximately 0800 and 0830 hours with breakfast). For all subjects, salivary samples for adrenal androgens and precursors will be collected in the early morning (0600 hours), at 6 hours after the morning glucocorticoid and study drug dose, and in the evening (within 30 minutes prior to the evening dose of glucocorticoid; if subject is not on an evening glucocorticoid dose, then prior to bedtime). The salivary samples at approximately 0800 hours prior to dosing and 3 and 6 hours after dosing should be collected at approximately the same time as any corresponding blood samples (as applicable). Any midday glucocorticoid dose that the subject is receiving should be taken after the 6-hour postdose blood and salivary samples are collected. During the study, the exact timing of the blood and saliva samples, and the morning glucocorticoid, mineralocorticoid, and/or study drug dose, may be adjusted by ±1 hour to accommodate site and subject schedules if needed, as long as the relative times between the sample(s) and the dose(s) are maintained.

From Week 4 until Week 28, the subject's glucocorticoid dose should be adjusted according to their androstenedione (A4) levels, with the goal to reach a target dose of 8 to 10 mg/m₂/day (in hydrocortisone dose equivalents adjusted for body surface area [BSA]) at Week 28, while A4 is controlled, i.e., ≤120% of the baseline value or ≤upper limit of normal (ULN), according to sex and either age (for Tanner stage 1) or pubertal stage (for Tanner stages 2 to 5). The calculation of glucocorticoid dose in hydrocortisone equivalents adjusted for BSA at Day 1 and Week 28 will be based on height and weight measurement at Day 1 and Week 28, respectively. BSA will be updated at Week 16 if height measurement is obtained at Week 16.

Glucocorticoid dose adjustments can occur in as few as 1 or up to 4 steps, depending on the starting and target glucocorticoid doses and the amount of dose adjustment at each step. Reductions in the glucocorticoid dose should follow the guideline of first reducing the most nonphysiologic glucocorticoid type and timing. The target glucocorticoid dose should be within the range of 8 to 10 mg/m²/day while A4 levels are controlled and as long as there is no evidence of glucocorticoid insufficiency. The dose could be lower than this range if the investigator considers this appropriate depending on practical issues considered in clinical practice related to available dosage strengths but will not be mandated to be lower than this range. Before any glucocorticoid dose reduction is implemented, the investigator will evaluate the subject for any symptoms suggestive of glucocorticoid insufficiency using a standardized checklist and will arrange for follow-up if needed after the dose reduction.

The first glucocorticoid dose adjustment step at approximately Week 6 (or when the Week 4 lab results are available) should be guided by the change in A4 at Week 4 from baseline. A suggested guideline is provided in the table below, but the exact amount adjusted may differ from this guideline based on practical issues considered in clinical practice related to available dosage strengths. The investigator should contact the subject/guardian once the Week 4 lab results are available in order to provide guidance on the amount of the first glucocorticoid dose adjustment. The first glucocorticoid dose adjustment step guidance is summarized in Table 1 below.

TABLE 1
First Glucocorticoid Dose Adjustment Guidance

| Percent Change from | Andro- | |
|---|---|---|
| Baseline in Serum | stenedione | Glucocorticoid Dose |
| Androstenedione | Level | Adjustment |
| Any increase | >ULN | Consider whether glucocorticoid |
| | | dose needs to be adjusted |
| Any increase | ≤ULN | Maintain current glucocorticoid |
| | | dose |
| No change^{a} | Any | Maintain current glucocorticoid |
| | | dose |
| Decrease of ≤20% | Any | 1 to 2 mg/m²/day glucocorticoid |
| | | dose decrease |
| Decrease of >20% | Any | 2 to 3 mg/m²/day glucocorticoid |
| to ≤40% | | dose decrease |
| Decrease of >40% | Any | 3 to 4 mg/m²/day glucocorticoid |
| | | dose decrease |

ULN = upper limit of normal
^{a}Within reasonable variability in the opinion of the investigator

A follow-up blood test (with salivary sample collection) should be arranged approximately 2 weeks later at Week 8 (at home or the study site).

If needed, subsequent glucocorticoid dose adjustment steps should occur when lab results are available (at approximately Week 10, Week 14, and Week 18) with follow-up blood and salivary tests at Week 12 (at home or the study site, if the glucocorticoid dose was previously modified), Week 16 (at home or the study site), and Week 20 (at home or the study site, if the glucocorticoid dose was previously modified). The target amount of glucocorticoid dose reduction at each step is approximately 1 to 4 mg/m²/day but should be guided by the A4 level at the preceding blood test as well as on practical issues considered in clinical practice related to available dosage strengths. The subsequent glucocorticoid dose adjustment step guidance is summarized in Table 2 below.

TABLE 2
Subsequent Glucocorticoid Dose Adjustment Guidance

| Blood Test (Including | |
|---|---|
| Androstenedione) | Glucocorticoid Dose Adjustment Step |
| Week 8 | Potential GC dose adjustment (if needed) at |
| | approximately week 10 (or when week 8 labs |
| | available) |
| Week 12 | Potential GC dose adjustment (if needed) at |
| | approximately week 14 (or when week 12 labs |
| | available) |
| Week 16 | Potential GC dose adjustment (if needed) at |
| | approximately week 18 (or when week 16 labs |
| | available) |
| Week 20 | Potential GC dose adjustment (if needed) at |
| | approximately week 22 (or when week 20 labs |
| | available) |

GC = glucocorticoid

At all visits, if A4 is >120% of baseline and >ULN, increase glucocorticoid dose as appropriate in order to maintain A4 control.

For the Week 28 visit, subjects should hold their morning glucocorticoid and study drug dose until after the predose blood samples are collected, with dosing to occur between approximately 0800 and 0830 hours with breakfast. For subjects ≥6 years of age with a body weight ≥20 kg, another blood sample will be collected approximately 3 hours after the morning glucocorticoid and study drug dosing. For all subjects, salivary samples will be collected over the course of the day: at approximately 0600 hours, at approximately 0800 hours prior to morning glucocorticoid and study drug dosing and 3 and 6 hours after dosing, and within 30 minutes prior to the evening dose of glucocorticoid (if subject is not taking evening glucocorticoid dose, then prior to bedtime). Any midday glucocorticoid dose that the subject is receiving should be taken after the 6-hour postdose salivary sample is collected.

### Open-Label Treatment Period (Week 28 to Week 52)

From the evening of the Week 28 visit (after all Week 28 assessments have been performed) until the morning of the Week 52 visit, all subjects will receive active study drug based on their Week 28 weight (Compound A; 25 mg b.i.d. via oral solution for subjects 10 to <20 kg, 50 mg b.i.d. via oral solution for subjects 20 to <55 kg, or 100 mg b.i.d. via oral capsules for subjects ≥55 kg) with breakfast and evening meals. Subjects and investigators will remain blinded to subjects' randomized treatment group assignment during the entire study. From Week 28 until Week 32, subjects should maintain a stable glucocorticoid regimen to the extent possible, except for stress dosing if needed for illness or other significant physical stress. A blood sample will be collected at Week 32 (at home or the study site).

For subjects who are on >11 mg/m²/day glucocorticoid dose (in hydrocortisone dose equivalents adjusted for BSA [based on weight and height at Week 28]) at Week 32, further adjustments in glucocorticoid dose should be made following guidelines similar to that used during the placebo-controlled period with the goal to reach a target dose of 8 to 10 mg/m²/day in hydrocortisone dose equivalents adjusted for BSA at Week 52, while A4 is controlled. The calculation of glucocorticoid dose in hydrocortisone equivalents adjusted for BSA at Week 52 will be based on height and weight measurement at Week 52. BSA will be updated at Week 40 if height measurement is obtained at Week 40.

The first glucocorticoid dose adjustment step during this period (if done) should be guided by the serum A4 change at Week 32 (compared with Week 28), after the subject has been on open-label active study drug as well as stable glucocorticoid regimen (to the extent possible) for 4 weeks. A suggested guideline is provided below but the exact amount adjusted may differ from this guideline based on practical issues considered in clinical practice related to available dosage strengths. The open label extension first glucocorticoid dose adjustment guidance is summarized in Table 3 below.

TABLE 3
Open Label Extension First Glucocorticoid
Dose Adjustment Guidance

| Percent Change from | Andro- | |
|---|---|---|
| Week 28 in Serum | stenedione | Glucocorticoid Dose |
| Androstenedione | Level | Adjustment |
| Any increase | >ULN | Consider whether glucocorticoid |
| | | dose needs to be adjusted |
| Any increase | ≤ULN | Maintain current glucocorticoid |
| | | dose |
| No change^{a} | Any | Maintain current glucocorticoid |
| | | dose |
| Decrease of ≤20% | Any | 1 to 2 mg/m²/day glucocorticoid |
| | | dose reduction |
| Decrease of >20% | Any | 2 to 3 mg/m²/day glucocorticoid |
| to ≤40% | | dose reduction |
| Decrease of >40% | Any | 3 to 4 mg/m²/day glucocorticoid |
| | | dose reduction |

ULN = upper limit of normal
^{a}Within reasonable variability in the opinion of the investigator

If the glucocorticoid dose is modified at approximately Week 34, a follow-up blood test (with salivary sample collection) should be arranged approximately 2 weeks later at Week 36 (at home or the study site).

If needed, subsequent glucocorticoid dose adjustments should occur at approximately Week 38 and Week 42 (or when lab results are available) with follow-up blood and salivary tests at Week 40 (at home or the study site) and Week 44 (at home or the study site, if the glucocorticoid dose was previously modified during the open-label treatment period). The target amount of glucocorticoid dose reduction at each step is approximately 1 to 4 mg/m²/day but should be guided by the A4 level at the preceding blood test as well as practical issues considered in clinical practice related to available dosage strengths. The open label extension subsequent glucocorticoid dose adjustment guidance is summarized in Table 4 below.

TABLE 4
Open Label Extension Subsequent Glucocorticoid
Dose Adjustment Guidance

| Blood Test (Including | |
|---|---|
| Androstenedione) | Glucocorticoid Dose Adjustment Step |
| Week 36 | Potential GC dose adjustment (if needed) at |
| | approximately week 38 (or when week 36 labs |
| | available) |
| Week 40 | Potential GC dose adjustment (if needed) at |
| | approximately week 42 (or when week 40 labs |
| | available) |
| Week 44 | Potential GC dose increase (if needed) at |
| | approximately week 46 (or when week 44 labs |
| | available) to maintain androstenedione control |

GC = glucocorticoid;
ULN = upper limit of normal

At all visits, if A4 is >120% of baseline and >ULN, increase glucocorticoid dose as appropriate in order to maintain A4 control.

For the Week 52 visit, subjects should hold their morning glucocorticoid and study drug dose until after the predose blood samples are collected, with dosing to occur between approximately 0800 and 0830 hours with breakfast. For subjects ≥6 years of age with a body weight ≥20 kg, another blood sample will be collected approximately 3 hours after the morning glucocorticoid and study drug dose. For all subjects, salivary samples will be collected over the course of the day: at approximately 0600 hours, at approximately 0800 hours prior to morning glucocorticoid and study drug dosing and 3 and 6 hours after dosing, and within 30 minutes prior to the evening dose of glucocorticoid (if subject is not taking evening glucocorticoid dose, then prior to bedtime). Any midday glucocorticoid dose that the subject is receiving should be taken after the 6-hour postdose salivary sample is collected.

### Open-Label Extension for Continued Compound A Access (Week 52 Onwards)

During the OLE, subjects will continue to receive Compound A at the appropriate dose based on their body weight at each study visit (25 mg b.i.d. if weight is 10 to <20 kg, 50 mg b.i.d. if weight is 20 to <55 kg, 100 mg b.i.d. if weight is ≥55 kg), unless the subject has inadequate efficacy (in the opinion of the investigator), in which case the Compound A dose can be increased by doubling the evening dose. Dose can be increased for inadequate efficacy once at the specified weight-based dose (e.g., 50 mg b.i.d. can be increased to 50 mg q.A.M. and 100 mg q.P.M.) but not further unless the subject crosses into the next weight category. If the investigator assesses the subject as having inadequate efficacy and in addition the subject has crossed into the next weight category, the Compound A dose should be increased first based on weight (not doubling the evening dose); efficacy should be assessed at the next scheduled study visit, and if efficacy remains inadequate the evening dose can be doubled at that time. If the Compound A dose is increased due to inadequate efficacy, a follow-up study visit should be performed approximately 1 month after the dose increase for efficacy, PK, and safety assessments. This follow-up study visit is not required for subjects who increase their dose due to crossing into the next weight category. Changes to the Compound A dose (whether based on crossing into the next weight category or based on inadequate efficacy) should generally only be made at scheduled study visits.

At Month 12, subjects taking Compound A 50 mg b.i.d. or higher may elect to switch the Compound A formulation (oral solution or capsule) based on preference, e.g., if the subject was receiving Compound A 50 mg b.i.d. oral solution from Week 28 to Month 12 but prefers capsule, they could switch to 50 mg b.i.d. capsule if remaining in the same weight category. Once this formulation preference is established at Month 12, the formulation should remain the same for the rest of the OLE (unless there is a tolerability issue, in which case the subject can revert back to the previous formulation, ideally within approximately 1 month).

During the OLE, subjects will have their glucocorticoid doses adjusted as appropriate and tolerated to achieve the lowest glucocorticoid dose that maintains adequate disease control (in the opinion of the investigator). The glucocorticoid dose reduction will not require dose reduction below 8 mg/m²/day hydrocortisone equivalents. After each glucocorticoid dose reduction, the investigator should contact the subject/guardian (within 2 weeks) to assess how the subject is tolerating the glucocorticoid dose reduction. In the setting of inadequate disease control, if the glucocorticoid dose is at or above the target, an increase in the glucocorticoid dose should generally be considered only after the Compound A dose has been maximized for the subject. Changes to the glucocorticoid and Compound A doses should generally be separated by at least 1 month in order to assess the effect of each change.

Starting at Month 12, study visits during the OLE will occur every 6 months. At each OLE study visit (including visits performed approximately 1 month after a dose increase for inadequate efficacy), subjects should hold their morning glucocorticoid and study drug dose until after the predose blood samples are collected, with dosing to occur between approximately 0800 and 0830 hours with breakfast. At Month 24 and every 12 months thereafter, for subjects ≥6 years of age with a body weight ≥20 kg, another blood sample will be collected approximately 3 hours after the morning glucocorticoid and Compound A dose. In addition, for all subjects, salivary samples will be collected over the course of the day at Month 24, every 12 months thereafter and at the early termination visit: at approximately 0600 hours, at approximately 0800 hours prior to morning glucocorticoid and study drug dosing and 3 and 6 hours after dosing, and within 30 minutes prior to the evening dose of glucocorticoid (if subject is not taking evening glucocorticoid dose, then prior to bedtime). Any midday glucocorticoid dose that the subject is receiving should be taken after the 6-hour postdose salivary sample is collected.

### Investigational Product, Dosage, and Mode of Administration:

Compound A (25 mg b.i.d. via oral solution for subjects 10 to <20 kg, 50 mg b.i.d. via oral solution for subjects 20 to <55 kg, or 100 mg b.i.d. via oral capsules for subjects ≥55 kg) with subjects' breakfast and evening meals (each dose separated by approximately 12 hours) from Day 1 to Month 12. During the OLE, the Compound A dose should be based on the subject's weight at the study visit (25 mg b.i.d. if weight is 10 to <20 kg, 50 mg b.i.d. if weight is 20 to <55 kg, 100 mg b.i.d. if weight is ≥55 kg), unless the subject has inadequate efficacy (in the opinion of the investigator), in which case the Compound A dose can be increased by doubling the evening dose. At Month 12, subjects participating in the OLE could elect to switch Compound A formulation based on preference. Each Compound A oral capsule contains 50 mg of Compound A. The oral solution contains 50 mg of Compound A per 1 mL and will be administered via a graduated oral dosing syringe. Further guidance on changes to Compound A dosing in the OLE is provided in Table 5 below.

TABLE 5
Guidance for Dose Adjustment During the Open Label Extension

| Type of Change in | | | |
|---|---|---|---|
| Compound A | | | |
| Dosing During | Applicable Study | | Additional |
| OLE | Visits | Examples | Guidance |
| Formulation switch: | Month 12/Week 52 | 50 mg b.i.d. | If there is a |
| oral solution versus | | solution in core | tolerability issue on |
| capsule, based on | | study but prefer | the new formulation |
| subject preference | | capsule → switch to | (e.g., difficulty |
| (only subjects | | 50 mg b.i.d. capsule | swallowing capsule), |
| receiving 50 mg | | at Month 12. | the subject can revert |
| b.i.d. or higher). | | | back to previous |
| | | | formulation (ideally |
| | | | within |
| | | | approximately 1 |
| | | | month). |
| | | | Once formulation |
| | | | preference is |
| | | | established at or |
| | | | within |
| | | | approximately 1 |
| | | | month of Month 12, |
| | | | formulation should |
| | | | remain the same for |
| | | | the rest of the OLE. |
| | | | The 25 mg b.i.d. |
| | | | dose regiment |
| | | | remains as solution |
| | | | (no option for 25 mg |
| | | | via capsule). |
| | | | Change in |
| | | | formulation may |
| | | | occur in conjunction |
| | | | with weight-based |
| | | | adjustment or |
| | | | increase for |
| | | | inadequate efficacy. |
| Weight-based | Month 12/Week 52 | 50 mg b.i.d. based | An increase in dose |
| adjustment in dose | and every 6 months | on weight 20 to <55 | based on new weight |
| when subject's body | thereafter during | kg in core study, | category should be |
| weight increases | OLE | which increases | done first before |
| such that subject | | to ≥55 kg in OLE → | increasing dose for |
| crosses into the next | | adjust dose to 100 | inadequate efficacy |
| weight category | | mg b.i.d. based on | if both conditions |
| 10 to <20 kg: 25 mg | | new weight category | apply. |
| b.i.d. | | (even if efficacy | If weight decreases |
| 20 to <55 kg: 50 mg | | assessed as | into a lower weight |
| b.i.d. ≥55 kg: | | inadequate at the | category, no dose |
| 100 mg b.i.d. | | same time). | adjustment is |
| | | | necessary unless |
| | | | there is a tolerability |
| | | | issue. |
| Dose increase for | Once per weight | Inadequate | If the Compound A |
| inadequate efficacy | category starting at | efficacy at Month 12 | dose is increased for |
| by doubling the | Month 12 and any | on 50 mg b.i.d. and | inadequate efficacy, |
| evening dose | subsequent visit | subject remains in | a study visit is |
| 25 mg b.i.d. → 25 | during the OLE | the same weight | performed |
| mg q.A.M., 50 mg | | category → increase | approximately 1 |
| q.P.M. | | to 50 mg q.A.M., | month after dose |
| 50 mg b.i.d. → 50 | | 100 mg q.P.M. | increase for efficacy, |
| mg q.A.M., 100 mg | | | PK, and safety |
| q.P.M. | | | assessments. |
| 100 mg b.i.d. → | | | If there is a |
| 100 mg q.A.M., 200 | | | tolerability issue |
| mg q.P.M. | | | with the Compound |
| | | | A dose increase, the |
| | | | dose can be |
| | | | decreased back to |
| | | | prior dose. |
| | | | If there is a need to |
| | | | decrease the |
| | | | Compound A dose |
| | | | back to the prior |
| | | | dose based on |
| | | | tolerability, the dose |
| | | | should not be |
| | | | increased again for |
| | | | inadequate efficacy |
| | | | during the rest of the |
| | | | OLE. |
| | | | If subject weight |
| | | | crosses into the next |
| | | | weight category after |
| | | | increasing for |
| | | | inadequate efficacy, |
| | | | adjust the Compound |
| | | | A dose for weight |
| | | | first. |

b.i.d. = twice daily;
OLE = open-label extension;
PK = pharmacokinetic;
q.A.M. = once in the morning;
q.P.M. = once in the evening

Criteria for evaluation include:

### Efficacy:

Hormone measurements: A4 (serum and saliva), 17-hydroxyprogeterone (17-OHP; serum and saliva), adrenocorticotropic hormone (ACTH) (plasma), luteinizing hormone (LH; serum), follicle-stimulating hormone (FSH; serum), testosterone (serum and saliva), cortisol (serum and saliva), plasma renin activity (PRA) (measured upright)
Daily glucocorticoid regimen expressed in hydrocortisone dose equivalents adjusted for BSA (mg/m²/day)
Body weight and body mass index (absolute and standard deviation score [SDS] units)
Growth (assessed as Bayley-Pinneau predicted adult height, height, and height velocity in absolute and SDS units; only in growing subjects)
Bone age based on x-ray (assessed in absolute and SDS units, also adjusted for chronologic age; only for subjects not at adult height)
Fasting lipid panel and homeostatic model assessment of insulin resistance (HOMA-IR) based on fasting glucose and insulin levels
Menstrual Cycle Questionnaire (only females who have undergone menarche and are not on hormonal or intrauterine device contraceptives)
Hirsutism (only for female subjects) and acne scales
Testicular ultrasounds (to detect adrenal rest tumors; only in male subjects)

### Patient and Caregiver Reported Outcomes:

EQ-5D-Y for ages 8 to 15 years; EQ-5D-5L for ages 16 to 17 years
Pediatric Quality of Life Instrument (PedsQL)
PedsQL Family Impact Module

### Pharmacokinetics:

Blood samples to evaluate plasma concentrations of Compound A and metabolites will be collected throughout the study.

### Other:

Study drug palatability/ease of administration (palatability will be assessed by subjects ≥6 years of age, and ease of administration will be assessed by caregivers for subjects <6 years of age)
Cytochrome P450 (CYP) 21A2 genotyping

### Safety:

Adverse events (AEs) (including events requiring glucocorticoid stress dosing)
Clinical laboratory tests (chemistry, hematology, coagulation, fasting glucose, urinalysis)
Vital signs
Physical examinations, including height, weight, and Tanner stage
6- or 12-lead electrocardiograms (ECGs)
Brief Psychiatric Rating Scale, Children's Version
Columbia-Suicide Severity Rating Scale (only for subjects ≥6 years of age)

### Study Endpoints and Statistical Analysis

The primary endpoint is the change from baseline to Week 4 in serum A4. The first key secondary endpoint is the change from baseline to Week 4 in serum 17-OHP. The second key secondary endpoint is the percent change from baseline to Week 28 in glucocorticoid daily dose (in hydrocortisone dose equivalents adjusted for BSA [mg/m²/day]), while Week 28 serum A4 is ≤120% of the baseline value or <ULN, according to sex and either age (for Tanner stage 1) or pubertal stage (for Tanner stages 2 to 5). These endpoints will be analyzed using an analysis of covariance model and will include treatment group (Compound A versus placebo), stratification factors used in the randomization and, as appropriate, baseline value. The overall type I error of 0.05 will be controlled by testing the primary, first key secondary, and second key secondary endpoints sequentially in this order.

Secondary endpoints will include:
The achievement of a reduction in glucocorticoid daily dose to physiologic levels (≤11 mg/m²/day in hydrocortisone dose equivalent adjusted for BSA) at Week 28, while Week 28 serum A4 is ≤120% of the baseline value or ≤ULN, according to sex and either age (for Tanner stage 1) or pubertal stage (for Tanner stages 2 to 5).
Change from baseline to Week 28 in body mass index SDS.
Change from baseline to Week 28 in mean 24-hour salivary 17-OHP.
Acceptability and palatability of the study drug at Week 4.
Ratio of the change in bone age from baseline to Week 28 to the change in chronologic age from baseline to Week 28 (in subjects not at adult height).
Change from baseline to Week 52 in Bayley-Pinneau predicted adult height SDS (in subjects not at adult height).

### Example 2: Liquid Formulation for Compound A

Table 6 shows a liquid formulation of Compound A as the free base (concentration of 50 mg/mL). A representative manufacturing process example for the preparation of the liquid formulation is shown in International Application Publication No. WO 2020/115555.

TABLE 6
Representative Liquid Formulation for Compound A (Free Base)

| | | Weight | % |
|---|---|---|---|
| Component | Function | (mg/mL) | (w/v) |
| Compound A, free base | Drug Substance | 50.0 | 5 |
| Labrafac ™ Lipophile WL1349 | Liquid Vehicle | to 1 mL | 74.58 |
| Labrafil ™ M 1944 CS | Surfactant | 200.0 | 20 |
| (PEG-5 Oleate) | | | |
| Butylated Hydroxytoluene | Antioxidant | 1.7 | 0.17 |
| Saccharin | Sweetener | 1.5 | 0.15 |
| FONA orange flavor | Flavor | 1.0 | 0.1 |
| (e.g., Orange Flavor 884.096 5 U) | | | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof, 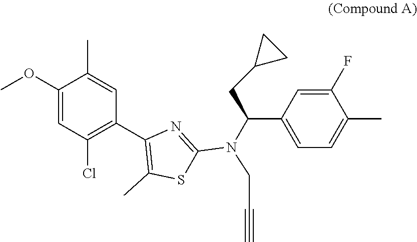
as an adjunct to glucocorticoid therapy in a pediatric subject having congenital adrenal hyperplasia (CAH), the method comprising: administering to the subject a first amount of Compound A, or a pharmaceutically acceptable salt thereof, for a first period of time, and subsequently administering to the subject a second amount of Compound A, or a pharmaceutically acceptable salt thereof, for a second period of time, wherein the second amount is administered in two doses consisting of a first dose and a second dose wherein the second dose is greater than the first dose.

2. A method of treating congenital adrenal hyperplasia (CAH) in a pediatric subject already being treated with glucocorticoid therapy, the method comprising: administering to the subject a first amount of 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof, 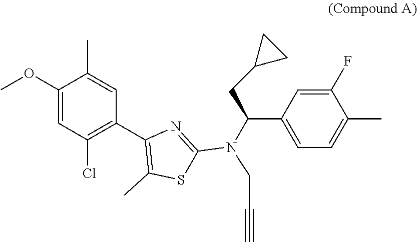
for a first period of time, and subsequently administering to the subject a second amount of Compound A, or a pharmaceutically acceptable salt thereof, for a second period of time, wherein the second amount is administered in two doses consisting of a first dose and a second dose wherein the second dose is greater than the first dose.

3. The method of claim 1 or 2, wherein the subject weighs greater than or equal to about 55 kg.

4. The method of claim 3, wherein the first amount is equivalent to about 100 mg Compound A free base b.i.d.

5. The method of claim 3 or 4, wherein the second amount is equivalent to about 100 mg of Compound A free base q.A.M. and about 200 mg Compound A free base q.P.M.

6. The method of any one of claims 3 to 5, wherein the first period of time is from about 1 week to about 1 year.

7. The method of any one of claims 3 to 6, wherein the second period of time is at least about 1 week.

8. The method of claim 1 or 2, wherein the subject weighs from about 20 kg to about 55 kg.

9. The method of claim 8, wherein the first amount is equivalent to about 50 mg Compound A free base b.i.d.

10. The method of claim 8 or 9, wherein the second amount is equivalent to about 50 mg of Compound A free base q.A.M. and about 100 mg Compound A free base q.P.M.

11. The method of any one of claims 8 to 10, wherein the first period of time is from about 1 week to about 1 year.

12. The method of any one of claims 8 to 11, wherein the second period of time is at least about 1 week.

13. The method of claim 1 or 2, wherein the subject weighs from about 10 kg to about 20 kg.

14. The method of claim 13, wherein the first amount is equivalent to about 25 mg Compound A free base b.i.d.

15. The method of claim 13 or 14, wherein the second amount is equivalent to about 25 mg of Compound A free base q.A.M. and about 50 mg Compound A free base q.P.M.

16. The method of any one of claims 13 to 15, wherein the first period of time is from about 1 week to about 1 year.

17. The method of any one of claims 13 to 16, wherein the second period of time is at least about 1 week.

18. The method of any one of claims 1 to 17, wherein the glucocorticoid therapy is a daily dose of a glucocorticoid administered to the subject.

19. The method of claim 18, wherein the daily dose of the glucocorticoid is administered once daily.

20. The method of claim 18, wherein the daily dose of the glucocorticoid is administered through multiple administrations.

21. The method of any one of claims 18 to 20, wherein the glucocorticoid is selected from hydrocortisone, prednisone, methylprednisolone, prednisolone, and dexamethasone, or a combination thereof.

22. The method of any one of claims 18 to 21, wherein the glucocorticoid is hydrocortisone.

23. The method of any one of claims 18 to 22, wherein prior to the administering of the second amount of Compound A, or a pharmaceutically acceptable salt thereof, the subject was receiving a glucocorticoid at a target dose.

24. The method of any one of claims 1 to 23, wherein prior to the administering of the second amount of Compound A, or a pharmaceutically acceptable salt thereof, the subject had inadequate efficacy.

25. The method of claim 24, wherein prior to the administering of the second amount of Compound A, or a pharmaceutically acceptable salt thereof, the subject had inadequate efficacy and was being administered a glucocorticoid at a target dose.

26. The method of claim 23 or 25, wherein the target dose of glucocorticoid treatment is ≤11 mg/m²/day hydrocortisone equivalents.

27. The method of claim 26, wherein the target dose of glucocorticoid treatment is about 8 mg/m²/day to about 10 mg/m²/day hydrocortisone equivalents.

28. The method of claim 27, wherein the target dose of glucocorticoid treatment is about 8 mg/m²/day hydrocortisone equivalents.

29. The method of any one of claims 25 to 28, wherein the subject had inadequate efficacy if their androstenedione levels are ≤120% of the subject's baseline or ≤upper limit of normal (ULN) for age and sex of the subject.

30. The method of any one of claims 1 to 29, wherein Compound A, or a pharmaceutically acceptable salt thereof, is the free base of Compound A.

31. The method of any one of claims 1 to 30, wherein Compound A, or a pharmaceutically acceptable salt thereof, is administered with food.

32. The method of any one of claims 1 to 31, wherein the CAH is classical CAH.

33. The method of any one of claims 1 to 31, wherein the CAH is non-classical CAH.

34. A compound which is 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine: 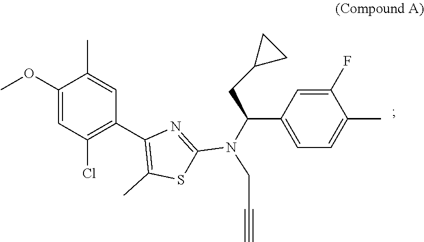
or a pharmaceutically acceptable salt thereof;
for use in a method of treating congenital adrenal hyperplasia (CAH) in a pediatric subject already being treated with glucocorticoid therapy, the method comprising:
(1) administering to the subject a first amount of Compound A, or a pharmaceutically acceptable salt thereof, for a first period of time, and
(2) subsequently administering to the subject a second amount of Compound A, or a pharmaceutically acceptable salt thereof, for a second period of time,
wherein the second amount is administered in two doses consisting of a first dose and a second dose wherein the second dose is greater than the first dose.

35. The compound or salt for use of claim 34, wherein the subject weighs greater than or equal to about 55 kg.

36. The compound or salt for use of claim 35, wherein the first amount is equivalent to about 100 mg Compound A free base b.i.d.

37. The compound or salt for use of claim 35 or 36, wherein the second amount is equivalent to about 100 mg of Compound A free base q.A.M. and about 200 mg Compound A free base q.P.M.

38. The compound or salt for use of any one of claims 35 to 37, wherein the first period of time is from about 1 week to about 1 year.

39. The compound or salt for use of any one of claims 35 to 38, wherein the second period of time is at least about 1 week.

40. The compound or salt for use of claim 34, wherein the subject weighs from about 20 kg to about 55 kg.

41. The compound or salt for use of claim 40, wherein the first amount is equivalent to about 50 mg Compound A free base b.i.d.

42. The compound or salt for use of claim 40 or 41, wherein the second amount is equivalent to about 50 mg of Compound A free base q.A.M. and about 100 mg Compound A free base q.P.M.

43. The compound or salt for use of any one of claims 40 to 42, wherein the first period of time is from about 1 week to about 1 year.

44. The compound or salt for use of any one of claims 40 to 43, wherein the second period of time is at least about 1 week.

45. The compound or salt for use of claim 34, wherein the subject weighs from about 10 kg to about 20 kg.

46. The compound or salt for use of claim 45, wherein the first amount is equivalent to about 25 mg Compound A free base b.i.d.

47. The compound or salt for use of claim 45 or 46, wherein the second amount is equivalent to about 25 mg of Compound A free base q.A.M. and about 50 mg Compound A free base q.P.M.

48. The compound or salt for use of any one of claims 45 to 47, wherein the first period of time is from about 1 week to about 1 year.

49. The compound or salt for use of any one of claims 45 to 48, wherein the second period of time is at least about 1 week.

50. The compound or salt for use of any one of claims 34 to 49, wherein the glucocorticoid therapy is a daily dose of a glucocorticoid administered to the subject.

51. The compound or salt for use of claim 50, wherein the daily dose of the glucocorticoid is administered once daily.

52. The compound or salt for use of claim 50, wherein the daily dose of the glucocorticoid is administered through multiple administrations.

53. The compound or salt for use of any one of claims 50 to 52, wherein the glucocorticoid is selected from hydrocortisone, prednisone, methylprednisolone, prednisolone, and dexamethasone, or a combination thereof.

54. The compound or salt for use of any one of claims 50 to 53, wherein the glucocorticoid is hydrocortisone.

55. The compound or salt for use of any one of claims 50 to 54, wherein prior to the administering of the second amount of Compound A, or a pharmaceutically acceptable salt thereof, the subject was receiving a glucocorticoid at a target dose.

56. The compound or salt for use of any one of claims 34 to 55, wherein prior to the administering of the second amount of Compound A, or a pharmaceutically acceptable salt thereof, the subject had inadequate efficacy.

57. The compound or salt for use of claim 56, wherein prior to the administering of the second amount of Compound A, or a pharmaceutically acceptable salt thereof, the subject had inadequate efficacy and was being administered a glucocorticoid at the target dose.

58. The compound or salt for use of claim 55 or 57, wherein the target dose of glucocorticoid treatment is ≤11 mg/m²/day hydrocortisone equivalents.

59. The compound or salt for use of claim 58, wherein the target dose of glucocorticoid treatment is about 8 mg/m²/day to about 10 mg/m²/day hydrocortisone equivalents.

60. The compound or salt for use of claim 59, wherein the target dose of glucocorticoid treatment is about 8 mg/m²/day hydrocortisone equivalents.

61. The compound or salt for use of any one of claims 56 to 60, wherein the subject had inadequate efficacy if their androstenedione levels are ≤120% of the subject's baseline or ≤upper limit of normal (ULN) for age and sex of the subject.

62. The compound or salt for use of any one of claims 34 to 61, wherein Compound A, or a pharmaceutically acceptable salt thereof, is the free base of Compound A.

63. The compound or salt for use of any one of claims 34 to 62, wherein Compound A, or a pharmaceutically acceptable salt thereof, is administered with food.

64. The compound or salt for use of any one of claims 34 to 63, wherein the CAH is classical CAH.

65. The compound or salt for use of any one of claims 34 to 63, wherein the CAH is non-classical CAH.

66. Use of a compound which is 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine: 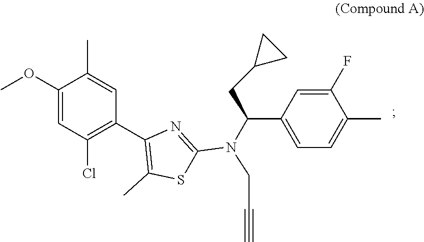
or a pharmaceutically acceptable salt thereof;
in the manufacture of a medicament for treating congenital adrenal hyperplasia (CAH) in a pediatric subject already being treated with glucocorticoid therapy, by a method comprising the steps:
(1) administering to the subject a first amount of Compound A, or a pharmaceutically acceptable salt thereof, for a first period of time, and
(2) subsequently administering to the subject a second amount of Compound A, or a pharmaceutically acceptable salt thereof, for a second period of time,
wherein the second amount is administered in two doses consisting of a first dose and a second dose wherein the second dose is greater than the first dose.

67. The use of claim 66, wherein the subject weighs greater than or equal to about 55 kg.

68. The use of claim 67, wherein the first amount is equivalent to about 100 mg Compound A free base b.i.d.

69. The use of claim 67 or 68, wherein the second amount is equivalent to about 100 mg of Compound A free base q.A.M. and about 200 mg Compound A free base q.P.M.

70. The use of any one of claims 67 to 69, wherein the first period of time is from about 1 week to about 1 year.

71. The use of any one of claims 67 to 70, wherein the second period of time is at least about 1 week.

72. The use of claim 66, wherein the subject weighs from about 20 kg to about 55 kg.

73. The use of claim 72, wherein the first amount is equivalent to about 50 mg Compound A free base b.i.d.

74. The use of claim 72 or 73, wherein the second amount is equivalent to about 50 mg of Compound A free base q.A.M. and about 100 mg Compound A free base q.P.M.

75. The use of any one of claims 72 to 74, wherein the first period of time is from about 1 week to about 1 year.

76. The use of any one of claims 72 to 75, wherein the second period of time is at least about 1 week.

77. The use of claim 66, wherein the subject weighs from about 10 kg to about 20 kg.

78. The use of claim 77, wherein the first amount is equivalent to about 25 mg Compound A free base b.i.d.

79. The use of claim 77 or 78, wherein the second amount is equivalent to about 25 mg of Compound A free base q.A.M. and about 50 mg Compound A free base q.P.M.

80. The use of any one of claims 77 to 79, wherein the first period of time is from about 1 week to about 1 year.

81. The use of any one of claims 77 to 80, wherein the second period of time is at least about 1 week.

82. The use of any one of claims 66 to 81, wherein the glucocorticoid therapy is a daily dose of a glucocorticoid administered to the subject.

83. The use of claim 82, wherein the daily dose of the glucocorticoid is administered once daily.

84. The use of claim 82, wherein the daily dose of the glucocorticoid is administered through multiple administrations.

85. The use of any one of claims 82 to 84, wherein the glucocorticoid is selected from hydrocortisone, prednisone, methylprednisolone, prednisolone, and dexamethasone, or a combination thereof.

86. The use of any one of claims 82 to 85, wherein the glucocorticoid is hydrocortisone.

87. The use of any one of claims 82 to 86, wherein prior to the administering of the second amount of Compound A, or a pharmaceutically acceptable salt thereof, the subject was receiving a glucocorticoid at a target dose.

88. The use of any one of claims 66 to 87, wherein prior to the administering of the second amount of Compound A, or a pharmaceutically acceptable salt thereof, the subject had inadequate efficacy.

89. The use of claim 88, wherein prior to the administering of the second amount of Compound A, or a pharmaceutically acceptable salt thereof, the subject had inadequate efficacy and was being administered a glucocorticoid at the target dose.

90. The use of claim 87 or 89, wherein the target dose of glucocorticoid treatment is ≤11 mg/m²/day hydrocortisone equivalents.

91. The use of claim 90, wherein the target dose of glucocorticoid treatment is about 8 mg/m²/day to about 10 mg/m²/day hydrocortisone equivalents.

92. The use of claim 91, wherein the target dose of glucocorticoid treatment is about 8 mg/m²/day hydrocortisone equivalents.

93. The use of any one of claims 88 to 92, wherein the subject had inadequate efficacy if their androstenedione levels are ≤120% of the subject's baseline or ≤upper limit of normal (ULN) for age and sex of the subject.

94. The use of any one of claims 66 to 93, wherein Compound A, or a pharmaceutically acceptable salt thereof, is the free base of Compound A.

95. The use of any one of claims 66 to 94, wherein Compound A, or a pharmaceutically acceptable salt thereof, is administered with food.

96. The use of any one of claims 66 to 95, wherein the CAH is classical CAH.

97. The use of any one of claims 66 to 95, wherein the CAH is non-classical CAH.
